SEQUENCE LISTING (31 sequences)

SEQ ID NO: 1 - protein, 18 aa
EWSPCSVTCGNGIQVRIK

SEQ ID NO: 2 - protein, 55 aa
PSDQHIKKYLNRIINSLSTEWSPCSVTCGNGIQVRIKPGSADKSPDQLDYANDIE

SEQ ID NO: 3 - protein, 55 aa
PSDKHIEQYLKKIKNSISTEWSPCSVTCGNGIQVRIKPGSANKDPDELDYENDIE

SEQ ID NO: 4 - protein, 55 aa
PSDKHIEEYLQTIQNSISTEWSPCSVTCGNGIQVRIKPGSAGKDPDELDYENDIE

SEQ ID NO: 5 - protein, 55 aa
PSDKHIETYLRKIKNSISTEWSPCSVTCGNGIQVRIKPGSANKDPDELDYENDIE

SEQ ID NO: 6 - protein, 16 aa
EWTPCSVTCGVGVRVR

SEQ ID NO: 7 - protein, 18 aa
EWSPCSVTCGSGIRARRK

SEQ ID NO: 8 - protein, 18 aa
EWSPCSVTCGKGVRMRRK

SEQ ID NO: 9 - protein, 18 aa
EWSPCSVTCGKGVRMRRK

SEQ ID NO: 10 - protein, 18 aa
EWSPCSVTCGSGIRARRK

SEQ ID NO: 11 - protein, 18 aa
EWSQCNVTCGSGIRVRKR

SEQ ID NO: 12 - protein, 18 aa
EWSQCSVTCGSGVRVRKR

SEQ ID NO: 13 - protein, 18 aa
EWSECSTTCDEGRKIRRR

SEQ ID NO: 14 - protein, 20 aa
PCSVTCGNGIQVRIKPGSAN

SEQ ID NO: 15 - protein, 20 aa
PASVTAGNGIQVRIKPGSAN

SEQ ID NO: 16 - protein, 20 aa
PXSVTXGNGIQVRIKPGSAN

SEQ ID NO: 17 - protein, 20 aa
CGNGIQVRIKPGSANKPKDE

SEQ ID NO: 18 - protein, 20 aa
AGNGIQVRIKPGSANKPKDE

SEQ ID NO: 19 - protein, 20 aa
PGSANKPKDELDYANDIEKK

SEQ ID NO: 20 - protein, 24 aa
CNVTCGSGIRVRKRKGSNKKAEDL

SEQ ID NO: 21 - protein, 20 aa
KPKHKKLKQPADGNPDPNAN

SEQ ID NO: 22 - protein, 19 aa
PGSANKPKDELDYANIELL

SEQ ID NO: 23 - protein, 13 aa
EWTPCSVTCGVGV

SEQ ID NO: 24 - protein, 14 aa
NKPKDQLDYQNDIQ

SEQ ID NO: 25 - protein, 17 aa
PSDKHIEQYLKKIKNSI

SEQ ID NO: 26 - protein, 17 aa
PSDQHIEKYLKRIQNSL

SEQ ID NO: 27 - protein, 10 aa
DKSKDQLNYA

SEQ ID NO: 28 - protein, 24 aa
CSVTCGSGIRVRKRKGSNKKAEDL

SEQ ID NO: 29 - protein, 6 aa
CSVTCG

SEQ ID NO: 30 - protein, 8 aa
CSVTCGRR

SEQ ID NO: 31 - protein, 8 aa
CSVTCGRR

(19) United States
(12) Patent Application Publication
(10) Pub. No.: US 2003/0119733 A1
(43) Pub. Date: Jun. 26, 2003

(54) Compositions and methods for inhibiting hepatocyte invasion by malarial sporozoites

(75) Inventors: Carla Cerami, New York, NY (US); Ute Frevert, New York, NY (US); Photini Sinnis, New York, NY (US); Victor Nussenzweig, New York, NY (US)

Correspondence Address: DARBY & DARBY P.C., 805 Third Avenue, New York, NY, 10022, US

(73) Assignee: New York University

(21) Appl. No.: 10/098,238

(22) Filed: Mar. 14, 2002

Related U.S. Application Data
(63) Continuation of application No. 08/119,694, filed on Sep. 10, 1993, now abandoned.
Application No. 08/119,694 is a continuation-in-part of application No. 07/947,033, filed on Sep. 17, 1992, now abandoned.

(51) Int. Cl. A61K 38/17; C07H 21/04; C12N 9/99; C12P 21/02; C12N 5/06

(52) U.S. Cl. 514/12; 435/184; 435/69.2; 435/320.1; 435/325; 536/23.2

(57) ABSTRACT

There is provided peptide and mimetic inhibitors for the binding of a circumsporozoite polypeptide to receptors of hepatocytes from malaria-susceptible mammals. Also contemplated is a method of inhibiting the binding of a malaria sporozoites to hepatocytes susceptible to sporozoite invasion. A peptide of Region II+ of the circumsporozoite protein is also provided, as is a method of targeting the delivery of substances to hepatocytes.

This application is a continuation-in-part of U.S. Ser. No. 07/947,033, filed Sep. 17, 1992.

The United States Government has rights to this invention by virtue of the following grants: Grant No. 5T32GM07308 from the National Institutes of Health; NIH5T 32CA9161-16; and Grant No. DPE-0453-A-00-5012-00 from the Agency for International Development.

### FIELD OF THE INVENTION

This invention is directed to compositions and methods for inhibiting hepatocyte invasion by malarial sporozoites. More specifically, the invention is directed to (a) ligands and mimetics thereof for the hepatocyte plasma membrane receptor for the circumsporozoite protein and peptides (and polypeptides) based on a portion of the circumsporozoite protein that constitutes an essential part of the specific ligand for this receptor; and (b) methods using such peptides to inhibit malaria sporozoite invasion of liver cells.

### BACKGROUND OF THE INVENTION

Malaria is transmitted by the bite of the Anopheles mosquito. Minutes after infection, sporozoites (the mosquito-hosted stage of the malarial parasite) enter hepatocytes of the susceptible mammal where they multiply by schizogony and develop into exoerythrocytic forms (“EEF”). Except in highly endemic areas, the number of parasites inoculated by a single mosquito is small, probably below 100, but malarial infection has high efficiency. This coupled with the uniqueness of the target (the victim's liver cells) suggests that hepatocyte invasion is receptor-mediated. However, neither the structure of the receptors nor that of the ligands had been elucidated.

The circumsporozoite protein, a malarial stage- and species-specific protein that uniformly covers the surface membrane of sporozoites isolated from mosquito salivary glands, constitutes one of the main proteins expressed by mature infective sporozoites, and would be a candidate ligand for a hepatic cell receptor if such a receptor existed.

The circumsporozoite (CS) protein has been extensively investigated (reviewed in Nussenzweig and Nussenzweig, Adv. Immunol. 45:283-334, 1989). The sequences of CS proteins from several malarial species have been elucidated and their main structural and antigenic properties which show substantial interspecies similarities have been described in Doolan et al., Infect. Immunol. 60:675-682, 1992; Lockyer et al., Mol. Biochem. Parasitol. 37:275, 1989; De La Cruz et al., J. Biol. Chem. 262:11925-11940, 1987; McCutchan et al., Science 230:1381-1383, 1985; Lal et al., Mol. Biochem. Parasitol. 30:291-294, 1988; Godson, et al., Nature 305:29-33, 1983; Galinsky et al., Cell 48:311-319, 1987; Lal et al., J. Biol. Chem. 263:5495-5498, 1988; Eichinger et al., Mol. Cell. Biol. 6:3965-3972, 1986; Lal et al., J. Biol. Chem. 262:2937-2940, 1987; and Hedstrom et al., WHO Bulletin OMS (Suppl.) 68:152-157, 1990. See also, U.S. Pat. No. 4,915,942; U.S. Ser. No. 07/370,241 filed Jun. 22, 1989, now allowed; U.S. Ser. No. 07/099,652, filed Sep. 21, 1987, now abandoned; and U.S. Ser. No. 07/864,172, filed Apr. 3, 1992.

All CS proteins contain (i) a species-specific immunodominant repeat domain encompassing about one-half of their molecule; (ii) two pairs of cysteines in the C-terminal region, and (iii) two relatively short stretches of conserved amino acid sequences flanking the repeat domain.

The N-terminal proximal conserved sequence (Region I) is the smaller of the two conserved regions and has been described in Dame, J. B. et al., Science 225:593-599, 1984. One group of investigators reported that peptides corresponding to Region I bind to hepatocytes and that antibodies against this region inhibit invasion (Aley, S. B. et al., J. Exp. Med. 164:1915-1921, 1986) but to the knowledge of the present inventors, there has been no follow up or independent confirmation of these studies.

The conserved sequence proximal to the C-terminal (Region II) surrounds the first pair of cysteines on the C-terminal side of the repeat domain. Region II was initially described by Dame et al., supra, but has now been redefined by the present inventors and as redefined will hereafter be referred to as Region II+. Region II+ is highly homologous to a cell-adhesion domain of thrombospondin (Prater et al., J. Cell. Biol. 112:1031-1040, 1991; Tuszynski, G. P. et al., Exp. Cell. Res. 182:473-481, 1989) as well as to regions of several other unrelated proteins such as properdin, von Willebrand factor, F-spondin, UNC-5, and antistasin, the latter being a leech anti-coagulant (Clarke, L. E. et al., Mol. Biochem. Parasitol. 41:269-280, 1990; Hedstrom, R. C. et al., WHO Bulletin OMS (Suppl.) 68:152-157, 1990; Robson, K. J. H. et al., Nature 335:79-82, 1988; Goundis, D. et al., Nature 335:82-85, 1988. Klar, A. et al., Cell 69:95, 1992; Leung-Hagesteyn, C., Cell 71:289, 1992). Region II as defined by Dame et al. did not prove to be immunogenic and was discarded as a candidate for a malaria vaccine. No function was attributed to it. CS regions adjacent to Region II, however, were shown to be immunogenic (See, e.g., U.S. Pat. No. 4,915,942). Region II+ was redefined based on extensive homology, considering not only P. knowlesi and P. falciparum (as Dame et al. had done) but also considering many other malarial species. See, Table 1 below.

Although the CS proteins have been extensively investigated and a large amount of information has been accumulated on their structure, immunological properties and evolution, their function remains unknown. The participation of the CS protein in hepatocyte invasion has been suggested by the observation that Fab fragments of monoclonal antibodies against the repeats inhibit sporozoite infectivity in vitro and in vivo. However, the ligand (if any) recognized by hepatocyte receptors did not seem to be in the repeats, in view of the fact that sporozoites of different species (the CS proteins of which have different repeat units) infect the liver of the same host. Moreover, immunization of hosts (especially human hosts) with synthetic repeat peptides abolished infectivity of sporozoites when relatively high levels of antibodies were elicited. Nevertheless, even small amounts of antibodies eliminated a large portion, but not all infected sporozoites. Antibodies to repeat peptides can attenuate the severity of subsequent malarial infection, and thus antibodies to repeat peptides have utility.

Malaria currently afflicts more than 200 million new human victims every year and accounts for nearly two million yearly deaths. In many parts of the world where malaria is endemic the parasites are resistant to all known chemotherapeutic drugs and there is evidence that resistance is spreading. Many investigators are currently involved in the development of vaccines against the sporozoite and the merozoite stage of malaria. Progress is being made but it is slow. As a result, malaria continues to threaten large numbers of the world's population. Malaria is most lethal to children and to travellers who, unlike adults from endemic areas, have no immunity to the disease. Partial immunity is acquired from continuous exposure to infected mosquitoes.

### OBJECTS OF THE INVENTION

Objects of the invention include the discovery of novel agents, including but not limited to ligands, receptor derived agents, and mimetics, and methods that are useful in inhibiting circumsporozoite protein binding to and sporozoite invasion of hepatocytes and in designing drugs and agents useful for the same purposes.

A further object of the invention includes the isolation and/or identification of the hepatocyte CS receptor, cleavage products thereof, and the corresponding ligands.

### SUMMARY OF THE INVENTION

In accordance with the present invention, there is provided a peptide inhibitor for the binding of a circumsporozoite polypeptide to receptors of hepatocytes from malaria-susceptible mammals. The inhibitor has an amino acid sequence selected from the group consisting of:

(i) Region II+ of a circumsporozoite protein, the Region II+ containing the subsequence CSVTCG;

(ii) fragments of the Region II+ containing at least a portion of the adhesion ligand for the receptors, the portion comprising at least one cysteine of the Region II+;

(iii) peptide constructs comprising (a) (i) or (ii) and (b) at least one other fragment of the amino acid sequence of the circumsporozoite protein, the constructs having no substantial ability to elicit the formation of antibodies recognizing the immunodominant epitope of the circumsporozoite protein. Peptide inhibitors also include dimers, multimers, and/or aggregates of any of the above, and particularly homo-dimers, -multimers, or -aggregates of any of the above, as well as structural, such as those having a similar three-dimensional configuration, or chemically functional mimetics thereof.

In another embodiment, a peptide inhibitor for the binding of a circumsporozoite polypeptide to basolateral plasma membrane of hepatocytes from malaria-susceptible mammals is provided. The inhibitor has an amino acid sequence selected from the group consisting of:

(i) Region II+ of a circumsporozoite protein, the Region II+ containing the subsequence CSVTCG;

(ii) fragments of the Region II+ containing at least a portion of the adhesion ligand for the receptors, the portion comprising at least one cysteine of Region II+;

(iii) peptide constructs comprising (a) (i) or (ii) and (b) at least one other fragment of the amino acid sequence of the circumsporozoite protein, the constructs having no substantial ability to elicit the formation of antibodies recognizing the immunodominant epitope of the circumsporozoite protein. Peptide inhibitors also include dimers, multimers, and/or aggregates of any of the above, and particularly homo-dimers, -multimers, and/or -aggregates of any of the above.

Additional inhibitors for the binding of a CS polypeptide or polypeptides as described above to a hepatocyte receptor in a malarial susceptible mammal are provided. These inhibitors comprise a cleavage product of a heparan sulfate proteoglycan from the surface of the hepatocyte. Structural and chemically functional mimetics thereof are provided as well.

Also contemplated is a method of inhibiting the binding of a circumsporozoite polypeptide to hepatocytes susceptible to sporozoite invasion comprising:

supplying to the environment of the hepatocytes the peptide inhibitor(s) or mimetic(s) above in an amount effective to inhibit the binding, no later than exposure of the hepatocytes to the circumsporozoite protein.

Peptides consisting essentially of Region II+ of the circumsporozoite protein and dimers, multimers and/or aggregates thereof, and mimetics thereof are also provided.

In an alternate embodiment, a method of delivering or targeting a substance to hepatocytes is provided. The substance is combined with the inhibitors above, and the resultant complex is administered to the individual.

### BRIEF DESCRIPTION OF THE DRAWINGS

FIG. 1: Is a schematic representation of various CS recombinant polypeptides.

FIG. 2: Shows photomicrographs illustrating the binding of CS polypeptides to sinusoids in liver sections or to human hepatocyte cell line HepG2. Binding is revealed with anti-repeat MAb 2A10 followed by a conjugate of rat anti-mouse IgG conjugated to fluorescein isothiocyanate.

Panel 2A: Binding of 12.5 μg/ml CS27IVC (control);

Panel 2B: Binding of 12.5 μg/ml falciparum-1;

Panel 2C: Inhibition of binding of CS27IVC by 250 μg/ml PfRII+;

Panel 2D: No inhibition of binding of CS27IVC by 500 μg/ml Pf3;

Panel 2E: Binding of CSFZ to HepG2.

FIG. 3: Panel A shows photomicrographs of the binding of CS polypeptides to human hepatocyte microvilli within the Space of Disse (arrows or lateral membranes of adjacent hepatocytes (arrowheads)), but no binding in bile canaliculi (BC) or endothelia cells (EC).

H: hepatocyte

N: nucleus of hepatocyte

S: sinusoid

E: erythrocyte

FIG. 3: Panel B shows a higher magnification of the Space of Disse of 3A showing aggregates of CS polypeptide (CS27IVC) binding to hepatocyte microvilli. Binding is revealed as in FIG. 2 but with gold instead of fluorescein.

FIG. 4: Shows photomicrographs of localized CS-binding sites in human and rat liver cells. Letters not defined above have the following significance: D=Disse space; L=lysosomes; K=Kupffer cell; *=contaminating cell organelle.

Panel A: binding of gold labelled CS27IVC human liver;

Panel B: binding of CS27IVC to rat liver—aggregated gold particles are shown under lysosomes of K but not on K surface;

Panel C: binding of CS27IVC to rat liver cell membrane fractions;

Panel D: non-binding of CS27IVC to rat liver mitochondrium and rough endoplasmic reticulum.

FIG. 5: Top panel shows the FPLC elution profile of CS27IVC following passage through a Superose column. Ordinate: OD×10⁻²; Abscissa: fraction number.

Bottom panel shows aggregates of CS in fraction 9 and monomers of CS in fractions 12-14. Molecular weight markers shown on top.

FIG. 6: A graph showing binding of CS to HepG2 cells. Fluorescence indicates amounts of bound protein.

FIG. 7: A graph of percent inhibition by RII+ peptides of the binding of CSFZ (Cys) to HepG2 cells as a function of RII+ concentration.

FIG. 8a: An electron micrograph of labeled human liver sections treated with chondroitinase.

D: Space of Disse;

Arrows: lateral domain of the hepatocyte plasma membrane;

L: liposomes;

M: mitochondrion

N: nucleus

Bars: 1 μm

FIG. 8b: An electron micrograph of labeled human liver sections treated with heparitinase.

D: Space of Disse;

Arrows: lateral domain of the hepatocyte plasma membrane;

L: liposomes;

M: mitochondrion

N: nucleus

Bars: 1 μm

FIG. 9: A graphic illustration of the inhibition of CS binding to HepG2 cells by glycosaminoglycans expressed as percent inhibition v. concentration of glycosaminoglycan.

FIG. 10: A graphic illustration of the inhibition of CS binding to HepG2 cells by heparitinase digestion expressed as percent inhibition v. concentration of heparitinase.

FIG. 11a: An electron micrograph of labeled rat kidney sections.

BC: Bowman capsule

Arrows: Laminae rarae

CL: capillary lumen

P: podocyte

BS: Bowman Space

DT: distal tube

BM: basement membrane

PT: proximal tube

L: lysosomes

N: nucleus

Bars: 1 μm

FIG. 11b: An electron micrograph of labeled rat binding sections.

BC: Bowman capsule

Arrows: Laminae rarae

CL: capillary lumen

P: podocyte

BS: Bowman Space

DT: distal tube

BM: basement membrane

PT: proximal tube

L: lysosomes

N: nucleus

Bars: 1 μm

FIG. 12: A photograph of an SDS-PAGE.

FIG. 13: A photograph of an SDS-PAGE.

FIG. 14: A graphic illustration of trypsin-release and CS-precipitation of HepG2 cell HSPG expressed as percent of total incorporated cpm released v. trypsin concentration.

FIG. 15: A graphic illustration of the ion exchange chromatography of trypsin-released HepG2 cell HSPG.

FIG. 16: A photograph of an SDS-PAGE.

FIG. 17: A photograph of an SDS-PAGE.

FIG. 18: A graphic illustration of molecular sieving chromatography of purified HSPG released by trypsin from HepG2 cells.

FIG. 19: A schematic representation of CS proteins.

FIG. 20: A graphic illustration of Vivax-1 and Vivax-2 binding to sulfatides and cholesterol-3-sulfate expressed as cpm v. concentration of peptide.

FIG. 21: A graphic illustration of the binding of Falciparum-2 to sulfatides but not to sulfatide analogues expressed as cpm v. concentration of peptide.

FIG. 22: A graphic illustration of the failure of Vivax-2 to bind the analogous of cholesterol-3-sulfate expressed as cpm v. concentration of steroid.

FIG. 23: A graphic illustration of the binding of P. berghei CS protein to sulfatide and cholesterol-3-sulfate expressed as cpm v. concentration of sporozoite equivalent.

FIG. 24: A Western Blot of CS proteins bound to sulfatides.

FIG. 25: A graphic illustration of the inhibition of CS protein binding to sulfatide and cholesterol-sulfatide by reduction and alkylation expressed as cpm v. concentration of sporozoite equivalent.

FIG. 26: A graphic illustration of the inhibition of CS protein binding to sulfatide and cholesterol-sulfatide by reduction and alkylation expressed as cpm v. concentration of peptide.

FIGS. 27A and B: Photographs of the anti-peptide serum recognition of glutaraldehyde fixed sporozoites.

FIG. 28A: A graphic illustration of the binding of CS27IVC to heparin sepharose.

FIG. 28B: A photograph of an SDS-PAGE.

FIG. 29: A graphic illustration of the CS27IVC functions obtained by incubation with fixed HepG2 cells.

FIGS. 30A, B, and C: Graphic illustrations of ligand clearance.

FIGS. 31A, B and C: Photomicrographs of mouse livers and kidneys stained with FITC labelled mAb 2A10.

FIGS. 32A and B: Photomicrographs of mouse liver after intravenous injection of CS27IVC.

### DETAILED DESCRIPTION OF THE INVENTION

All literature, patents, applications and other published documents cited herein are incorporated by reference in their entirety. In case of conflict, however, the present disclosure controls.

The term peptide as used herein includes polypeptides and the term polypeptides as used herein includes peptides.

Although the structure of the CS receptor is of importance in drug design, one of the advantages of the present invention is that knowledge of the receptor structure is not required. The hepatocyte CS receptor and the corresponding ligand can serve as a basis for rational drug design and DNA or drug delivery. For example, the incorporation of the Region II+ amino acid sequence into the envelope protein of a recombinant virus may enhance its capture by hepatocytes.

The peptides or constructs (which include the dimers, multimers, or aggregates described above) of the invention should have no substantial ability to elicit the formation of antibodies recognizing the immunodominant epitope of CS that would diminish the effectiveness of the peptides or constructs. However, these peptides or constructs may be recognized by antibodies raised by conjugating the peptides or constructs to a separate immunogenic component.

CS Binding and Inhibition

Materials

Recombinant P.vivax and P.falciparum proteins and polypeptide fragments thereof can be made, e.g. in accordance with now well-known recombinant techniques, see, e.g., Barr, P. J. et al., J. Exp. Med. 165:1160-1171, 1987 and U.S. Pat. Nos. 4,997,647 and 4,880,734 and European Patent Publication No. 460716. Their sequences as well as other recombinant methods for making them have been published in Dame, J. B. et al., Science 225:593-599, 1984; and McCutchan, T. F. et al., Science 230:1381-1383, 1985. Other CS sequences have been published in Doolan, D. L. et al., Infect. Immunol. 60:675-682, 1992, Lockyer, M. J. et al., Mol. Biochem. Parasitol. 37:275, 1989; De La Cruz, V. F. et al., J. Biol. Chem. 262:11935-11940, 1987; Galinsky, M. R. et al., Cell 48:311-319, 1987; Eichinger, D. J. et al., Mol. Cell. Biol. 6:3965-3972, 1986, Lal, A. A. et al., Mol. Biochem. Parasitol. 30:291-294, 1988; and Hedstrom, R. C. et al., WHO Bulletin OMS (Suppl.) 68:152-157, 1990.

The following peptide fragments of CS proteins were used in experiments:

| | |
|---|---|
| Vivax-2: | Includes the entire N-terminal moiety of P. vivax |
| | CS protein, the repeats Region II+ (as redefined |
| | by the present inventors) and 11 amino acid |
| | residues downstream from the end of the repeats |
| | through a leucine residue. Vivax-2 has no free |
| | sulfhydryl groups as determined by Ellman reac- |
| | tion. |
| Falciparum-2: | The corresponding fragment of P. falciparum CS |
| | protein, also terminating at the leucine residue |
| | (P. falciparum CS residues 43-391). Falciparum-2 |
| | also has no free sulfhydryl groups as determined |
| | by Ellman reaction. |
| Vivax-1: | Same as Vivax-2 minus most of Region II+, termi- |
| | nating with the proline residue immediately |
| | preceding the first cysteine of Region II+. |
| Falciparum-1: | Same as Falciparum-2 minus most of Region II+, |
| | terminating with the proline residue preceding |
| | the first cysteine of Region II+ (P. falciparum |
| | residues 43-348). |

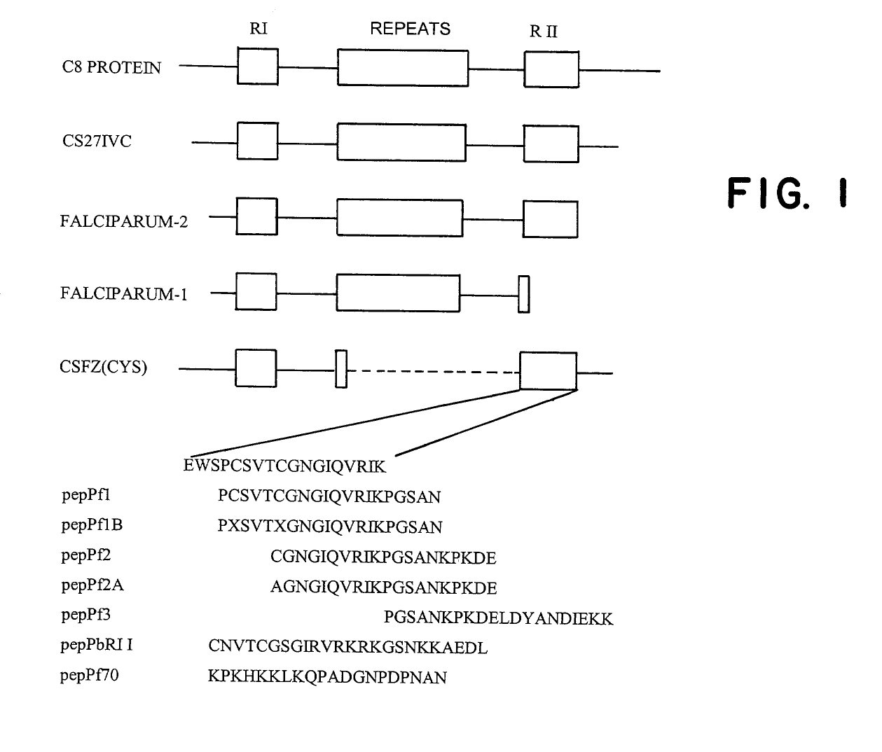

See FIG. 1 for more details on the foregoing polypeptides

| | |
|---|---|
| Region II+: | Region II in P. falciparum has been redefined by |
| | the present inventors as: |
| | E-W-S-P-C-S-V-T-C-G-N-G-I-Q-V-R-I-K |

The corresponding Regions II+ for other malarial species and the extensive homology among them are shown in Table 1 below.

| | |
|---|---|
| SPECIES | SEQUENCE |

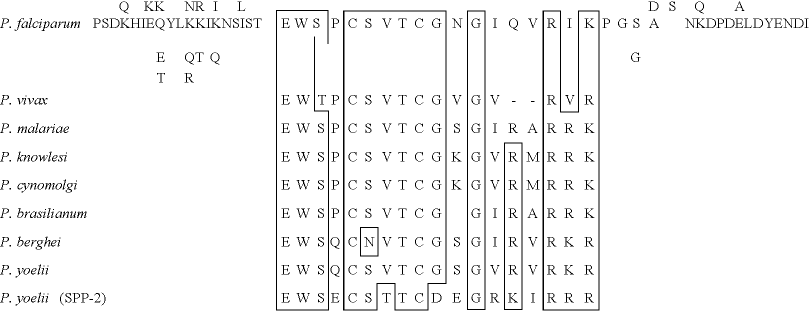

The conserved motif EWXXCXVTCGXGXXXRXK/R, encompassing a hepatocyte ligand for malaria sporozoites, is contained in the C-terminus of the CS protein. The recognition by the liver receptor requires the presence of the cluster of positively charged amino acids RXK/R at the N-terminus, and these peptides (RXK/R) specifically inhibit host cell invasion by sporozoites and binding of CS to its liver receptor.

In addition, the following peptides and polypeptides were made by recombinant DNA methodology:

| | |
|---|---|
| CS27IVC: | Consists from N- to C-terminal of residues 27- |
| | 123 plus (NANPNVDP)₃ plus (NANP)₂₁ plus residues |
| | 300-411 of the P. falciparum CS protein. See, |
| | Takacs et al., J. Immunol. Meth. 143:231-240, |
| | 1991; Hochuli et al., J. Chromat. 411:177-184, |
| | 1987. |
| CSFZ (Cys): | Consists of residues 27-123 plus NANP (a single |
| | instance) plus residues 300-411 of P. falciparum |
| | CS protein. |

In addition, the following P.falciparum derived peptides were synthesized as described in Houghten, R. A., Proc. Nat'l. Acad. Sci. USA 82:5131-5135, 1985.

| | |
|---|---|
| Pf1 | PCSVTCGNGIQVRIKPGSAN |
| Pf1A | PASVTAGNGIQVRIKPGSAN |
| Pf1B | PXSVTXGNGIQVRIKPGSAN |
| Pf2 | CGNGIQVRIKPGSANKPKDE |
| Pf2A | AGNGIQVRIKPGSANKPKDE |
| Pf3 | PGSANKPKDELDYANDIEKK |
| PbRII+ | CNVTCGSGIRVRKRKGSNKKAEDL |
| Pf70 | KPKHKKLKQPADGNPDPNAN |
| Pf4 | PGSANKPKDELDYANIEKK |
| Pf1: | contains most of P. falciparum Region II+. |
| Pf1A: | Identical to Pf1 except alanine residues |
| | are substituted for the two cysteines. |
| Pf1B: | Identical to Pf1 except the sulfhydryl |
| | groups on both cysteines have been blocked |
| | with acetamide groups. |
| Pf2: | Contains most of Region II+. |
| Pf2A: | Identical to Pf2 except the first cysteine |
| | has been replaced by an alanine residue. |
| Pf25C: | A scrambled version of Pf2. |
| Pf3: | Contains only a C-proximal moiety of |
| | Region II+. |
| PbRII+: | Contains Region II+ from P. berghei. |
| Pf70: | Contains most of P.falciparum Region I. |
| Pf4: | Contains only a C-proximal moiety of |
| | Region II+ except for a single amino acid |
| | deletion. |

Antibodies

Monoclonal antibody 2A10 (prepared according to Nardin, E. H. et al., J. Exp. Med. 156:20-30, 1982) is directed against an epitope contained in the repeat—(NANP)ₙ—region of P.falciparum CS protein and recognizes the amino acid sequence PDPNANPN found 5′ of Region II in the repeat-less recombinant polypeptide CSFZ (Cys) (Burkot, T. R. et al., Parasite Immunol. 13:161-170, 1991).

Monoclonal antibody 2E6 reacts with the liver stage of P.berghei. Such antibodies can be made by methods known to those skilled in the art.

Polyclonal antisera against P.berghei CS Region II were raised by immunizing rabbits with peptide PbRII coupled to key hole limpet hemocyanin with glutaraldehyde. A rabbit was immunized once with 500 μg of the peptide-adjuvant conjugate in complete Freund's adjuvant and then boosted monthly (four times) with the same amount of conjugate in incomplete Freund's adjuvant. The antisera recognize the peptide PbRII+ dried onto plastic wells, and this binding is inhibited by soluble PbRII+. The difficulty of making such antisera testifies to the non-immunogenic character of Region II+.

The invention is described in more detail below by reference to specific examples, which are only illustrative and not limiting in nature.

### EXAMPLE 1

Staining of Frozen Tissue Sections

The procedure was carried out essentially as described by Dukor P. et al., Proc. Natl. Acad. Sci. USA 67:991-997, 1970 and Imai Y. et al., J. Cell. Biol. 111:1225-1232, 1990. Rats were euthanized with CO₂. Small pieces of the liver, spleen, heart, brain and lung were removed, snap frozen in liquid nitrogen, embedded in Tissue Tek O.C.T. (Miles, Inc., Naperville, Ill.), and cut into 5 μm sections. Sections were dried for 30 min., fixed for 10 min. in 4% paraformaldehyde, rinsed three times with phosphate-buffered saline (PBS), and either used immediately or stored at 4° C. in 1% BSA, 0.5% Tween, PBS (BSA/TPBS). After blocking with 100 mM glycine (pH 7.2) and with BSA/TPBS, the sections were sequentially incubated at 37° C. with recombinant proteins for 1 hr, 10 μg/ml MAb 2A10 for 45 min., and rat anti-mouse immunoglobulin conjugated to fluorescein isothiocyanate (Boehringer Mannheim, Indianapolis, Ind.) for 45 min. The slides were counterstained for 10 min. with 0.3% Evans blue, rinsed extensively in PBS, and finally observed under a fluorescence microscope. For peptide inhibition experiments, the sections were first incubated with the peptide at various concentrations in BSA/TPBS for 1 hour at 37° C., washed six times with BSA/TPBS, and then stained with 2.5 μg/ml CS recombinant protein as described above. In a few experiments, the tissues were fixed for 10 min. with methanol, acetone, or 4% paraformaldehyde containing 0.5% glutaraldehyde.

### EXAMPLE 2

Isolation of Hepatocyte Membranes

Fractionation of rat liver cells was performed as described by Hubbard A. L. et al., J. Cell. Biol. 96:217-229, 1983. In brief, perfused rat livers were homogenized and subjected to sucrose gradient centrifugation. The membrane preparation and the pellet, consisting mostly of mitochondria and rough endoplasmic reticulum, from the final centrifugation step were processed for ultrastructural examination.

### EXAMPLE 3

Electron Immunomicroscopy

Rat or mouse liver tissue or hepatocyte subcellular fractions from Example 2 were fixed in PBS containing 1% glutaraldehyde (grade 1, Sigma, St. Louis, Mo.) and 4% paraformaldehyde (Kodak, Rochester, N.Y.), dehydrated in ethanol, and embedded in LR White (Polysciences, Warrington, Wash.). (Frevert et al., Infect. and Immun. 60:2349-2360, June 1992.) Normal human liver was embedded in Lowicryl K4M (Ted Pella, Redding, Calif.). Ultrathin sections were labelled by incubating them sequentially with 10-50 μg/ml CS27IVC, CSFZ (Cys), Falc-2, or Falc-1 for 30 min.; 15 μg/ml MAb 2A10 for 30 min.; protein A-gold 15 nm (PAG15, 1:30; Amersham, Arlington, Ill.) or goat anti-mouse IgG gold 10 nm (GAM10, 1:30; Amersham) for 30 min. Control specimens were incubated in the absence of CS and only with the gold conjugates. Photographs were taken with a Philips EM 301 electron microscope.

### EXAMPLE 4

HeDG2 Cell Binding Assay

For indirect immunofluorescence, HepG2 cells (ATCC number HB8065, Rockville, Md.; Knowles, B. P., et al., Science 209:497-499, 1980) were grown on slides (Cel-Line Associates, Inc., Newfield, N.J.) overnight in minimum essential medium with 10% fetal calf serum (FCS-MEM; GIBCO, Grand Island, N.Y.), 1 mM L-glutamine (GIBCO), 3 mg/ml glucose (Sigma), 1×nonessential amino acids (GIBCO), 50 μg/ml penicillin, and 100 μg/ml streptomycin (GIBCO). For the enzyme-linked immunosorbent assay, 10⁵ HepG2 cells were deposited in 96-well Falcon tissue culture plates (Becton Dickinson, Oxnard, Calif.) and grown for 24 hr. in FCS-MEM. The cells were fixed with 4% paraformaldehyde, washed three times with PBS, and stored at 4° C. in BSA/TPBS until use. Before the experiments, plates were blocked for 2 hr. at 37° C. with 1% gelatin, 0.05% Tween in PBS (pH 7.4) (gelatin/TPBS). The cells were sequentially incubated at 37° C. with 50 μl of recombinant protein diluted in gelatin/TPBS for 1 hr. MAb 2A10 at a concentration of 10 μg/ml for 30 min. and goat anti-mouse immunoglobulin conjugated to alkaline phosphatase (Boehringer Mannheim) for 30 min. Bound enzyme was revealed by the addition of the fluorescent substrate, 1 mM 4-methylumbelliferyl phosphate in 100 mM Tris-HCl, 100 mM NaCl, and 5 mM MgCl₂ (pH 9.5). After 15 min., fluorescence was read in a Fluoroskan II plate reader (Flow Lab Inc., McLean, Va.) with excitation filter 350 nm and emission filter 460 nm. In the peptide inhibition experiments, wells were first incubated for 1 hr. at 37° C. with peptides diluted in gelatin/TPBS, washed three times with gelatin/TPBS, and then incubated with recombinant CSFZ (Cys) at a concentration of 2.5 μg/ml for 1 hr. at 37° C. The bound CSFZ (Cys) was revealed as described below.

### EXAMPLE 5

FPLC Analysis of Recombinant CS Polypeptide

To separate monomers from multimers of recombinant CS, the preparations were subjected to molecular sieving chromatography on an FPLC apparatus (Pharmacia, Piscataway, N.J.). One hundred micrograms of CS27IVC in a phosphate buffer containing 150 mM NaCl (pH 7.2) was injected into a Superose 12 sizing column (Pharmacia). The protein was eluted in the same buffer using a flow rate of 0.2 ml/min. A high molecular weight fraction (69 kDa) co-eluted with thyroglobulin. The monomeric form of the protein eluted between amylase (molecular weight=200 kDa) and BSA (molecular weight=66kDa).

### EXAMPLE 6

Western Blotting of Recombinant CS Polypeptides

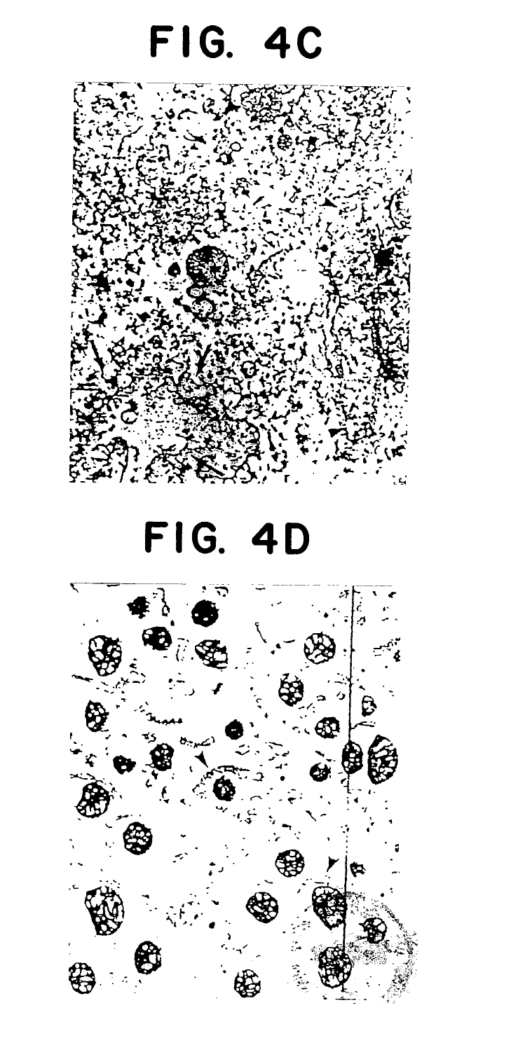

Western blotting was conducted as described by Towbin, H. T. et al., Proc. Natl. Acad. Sci. USA 76:4350-4354, 1979. Aliquots of the fractions obtained from the FPLC analysis were run on a 7.5% SDS-polyacrylamide gel under nonreducing conditions and electrophoretically transferred to Immobilon membrane (Millipore Corporation, Bedford, Mass.). The nylon membrane was blocked for 30 min. with BSA/TPBS, incubated 1 hr. with 15 μg/ml MAb 2A10, washed three times with 0.5% Tween/PBS, and incubated with goat anti-mouse antibody coupled to alkaline phosphatase (Sigma). The bound enzyme was developed with bromochlorophenol blue and nitrotetrazolium blue. The results are shown in FIG. 5 bottom panel which illustrate that fraction 9 contained polymeric forms of the CS protein and fractions 12, 13 and 14 contained mainly monomers.

### EXAMPLE 7

Preparation of Falciparum-1 Aggregates

The methods described by Lambert, J. M. et al., Biochemistry 17:5406-5416, 1978 were followed. Falciparum-1 at a concentration of 1 mg/ml was incubated with 250 mM Traut's reagent (Pierce Chemical Co., Rockford, Ill.) in 50 mM triethylamine-hydrochloric acid (TEA-HCl) (pH 8.0), 1 mM Mg(Ac)₂, and 50 mM KCl for 20 min. on ice. Traut's reagent (0.5 M stock) was prepared immediately before use in a solution containing equal volumes of 1 M TEA-HCl and 1 M TEA-free base. The sulfhydryl-containing falciparum-1 was oxidized in ambient air at 4° C. overnight. The presence of aggregated disulfide-linked falciparum-1 was confirmed both by chromatography in an FPLC sizing column and by Western blotting as described above.

### EXAMPLE 8

Sporozoite Infection Assay

Assays were conducted according to the methods described by Hollingdale, M. R. et al.. Science 213:1021-1022, 1981. and Sinden, R. E., WHO Bulletin MOS (Suppl.) 68:115-125, 1990. HepG2 cells were plated in FCS-MEM at a density of 0.5×10⁶ cells/ml in 8-chamber slides (4808 Lab-tek, Naperville, Ill.) 24 hr. before each experiment. For the peptide inhibition experiments, sporozoites were resuspended in FCS-MEM alone or FCS-MEM containing 500-125 μg/ml peptide. For antibody inhibition experiments, sporozoites were preincubated in FCS-MEM alone, or FCS-MEM containing anti-Region II IgG, or with preimmune sera IgG for 30 min. at 4° C. Sporozoites (5×10⁴), in a volume of 100 μl, were added to each well. The medium was replenished after 2 hr. and changed after 18 and 28 hr. Each point was performed in quadruplicate. In experiments designed to evaluate the toxicity of the peptides and antisera, sporozoites were first incubated with the HepG2 cells for 2 hr in FCS-MEM. FCS-MEM containing the peptide at a concentration of 250 μg/ml (or the IgGs at a concentration of 700 μg/ml) was then added to the cultures, and incubation proceeded for two additional hours. As above, FCS-MEM was changed after 18 and 28 hr. All cultures were fixed with cold methanol containing 0.3% H₂O₂ after 48 hr. Wells were blocked with BSA/TPBS, incubated for 45 min. with 10 μg/ml MAb 2E6 directed against the exoerythrocytic forms of the parasite, washed three times with BSA/PBS, incubated for 45 min. with goat anti-mouse immunoglobulin conjugated to horse-radish peroxidase (Accurate Chemical and Scientific Corp., Westbury, N.Y.), and washed three times with BSA/PBS. Bound enzyme was revealed with 1 mg/ml 3,3′-diaminobenzidine in 0.05M Tris (pH 7.6), 0.01% H₂O₂. The number of exoerythrocytic forms in 20 fields was counted in a double-blind fashion under a 20× light microscope objective.

### EXAMPLE 9

Binding of CS in the Liver

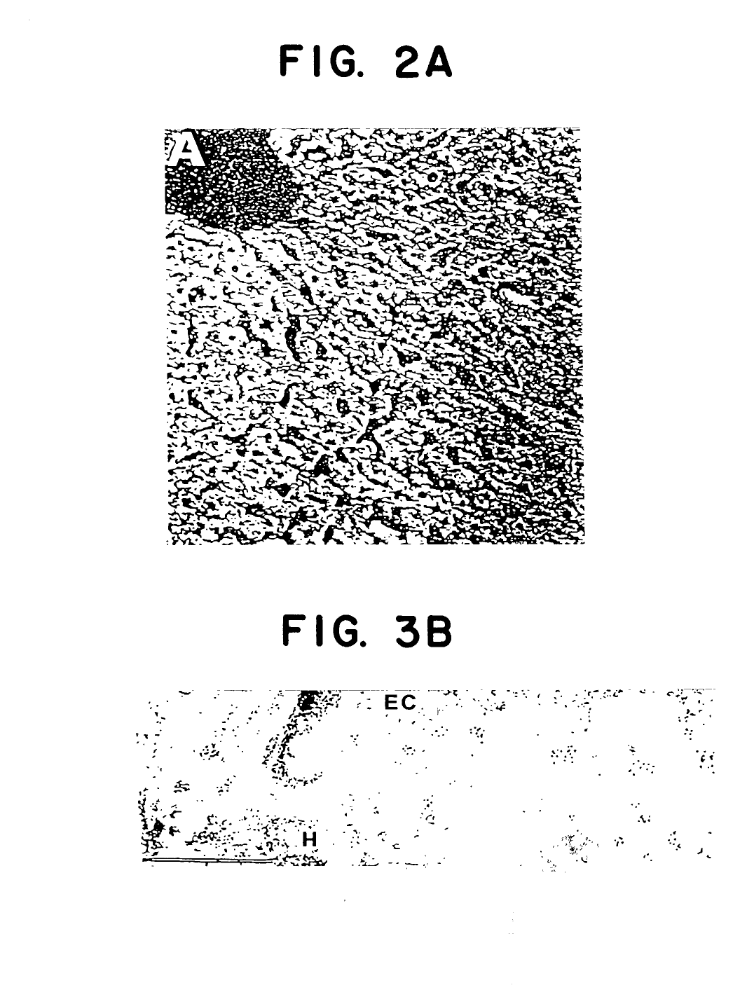

Frozen sections of various rat organs were incubated with recombinant CS27IVC polypeptide. Tissue-bound CS was revealed with MAb 2A10 and fluorescence microscopy. Strong staining was observed in liver sections with concentrations of CS27IVC within the range 50-5 μg/ml. The results are shown in FIG. 2 Panel 2A. The staining closely followed the sinusoidal spaces of the hepatic lobules, indicating CS binding to the hepatocyte membrane. Other areas of the liver sections and sections of other organs (spleen, lung, heart or brain) were not stained. Control sections (incubated with CS27IVC in the absence of antibody or vice versa) were not stained either.

The same pattern was observed using air-dried (instead of frozen) sections or in sections fixed with various fixtures (4% p-formaldehyde alone or in combination with 0.5% glutaraldehyde, acetone or methanol).

To define the region of the CS that mediates the perisinusoidal staining various other recombinant constructs were also used in the frozen section assay. The results were as follows:

CSFZ (Cys) (which contains Region II and Region I but only one repeat copy) bound with the same pattern as CS27IVC;

Falciparum-2 (which contains Region I and II as well as the repeats) bound to the liver sections at 25 μg/ml;

Peptides, PbRII+, Pf1 and Pf2 all containing substantially Region II+ inhibited the binding of CS27IVC with the inhibition being complete at 250 μg/ml of peptide PbRII+.

Recombinant polypeptide Falciparum-1 which contains Region I and the repeats but not Region II+ did not bind to liver sections even at concentrations as high as 250 μg/ml.

Peptides Pf3, Pf4, Pf1A, Pf1B, Pf2A, Pf25C and Pf70 all failed to inhibit the binding of CS27IVC to liver sections even at concentrations as high as 500 μg/ml (Pf3).

These results indicate that both certain amino acids and their sequence within Region II+ are essential for binding to liver sections. The presence of two cysteines, for example, is important. Omission of N-terminal CSVT abrogates a substantial portion of the peptides' inhibitory ability. In fact, as will be shown below, the sequence CSVTCG (and its variants in Table 1) appears to be important for inhibition.

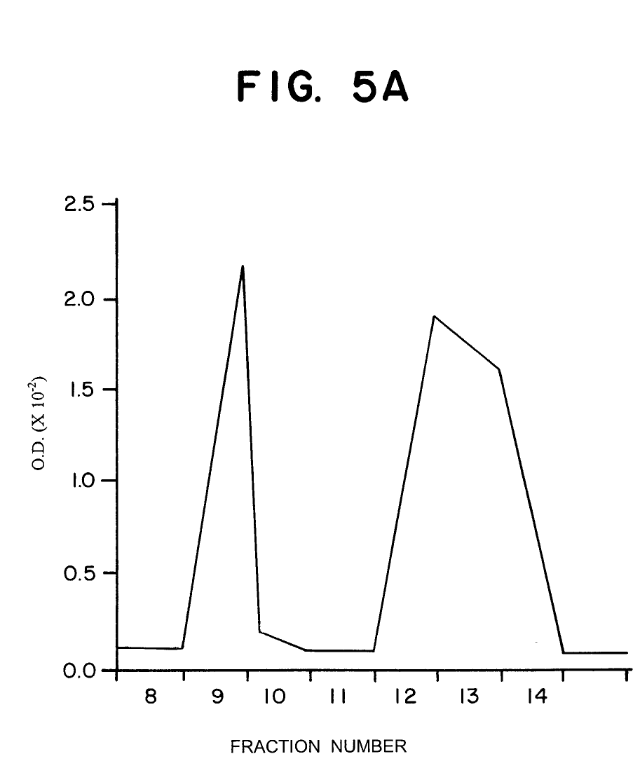

In order to quantify more accurately the effectiveness of various peptides in inhibiting the CS27IVC binding to hepatocytes additional experiments were performed using the human hepatoma cell line HepG2. This cell line is invaded readily by P.berghei sporozoites. Indirect immunofluorescence revealed that HepG2 cells bound recombinant CSFZ (Cys) but not Falciparum-1. Incubation of HepG2 cells immobilized on the bottom of microtiter plates with increasing concentrations of CSFZ (Cys) showed that CSFZ (Cys) binds to these cells in a dose-dependent and saturable manner. The results, expressed in fluorescence units v. protein concentration are depicted in FIG. 6. The open circles represent binding of CSFZ (Cys); the black circles represent binding of Falciparum-1 (control). Saturation is projected in FIG. 6 at about 2.5 μg/ml CSFZ (Cys).

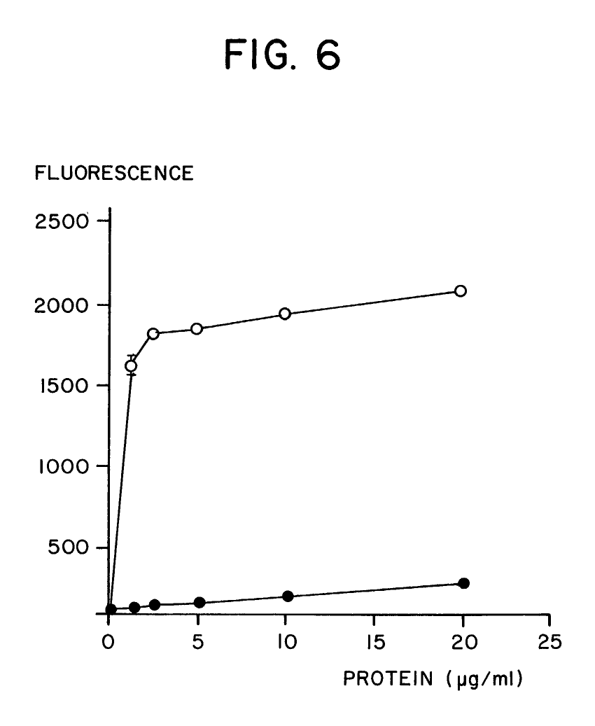

The same assay system was used to evaluate the ability of the various synthetic peptides to inhibit the binding of CSFZ (Cys) to the HepG2 cells. Paraformaldehyde-fixed immobilized HepG2 cells were pre-incubated with peptides at concentrations between 0 and 250 μg/ml, washed, and incubated with CSFZ (Cys) at 2.5 μg/ml. After washing, the cells were incubated with MAb 2A10 followed by anti-mouse IgG conjugated to alkaline phosphatase. Bound enzyme was revealed by a fluorescent substrate, methylumbelliferyl phosphate. Each peptide concentration was assayed in triplicate wells and the means of the fluorescent reading were calculated. The results were the same as in the frozen section experiments: Pf1 and Pf2 both inhibited the CSFZ (Cys) binding to HepG2 cells. Fifty percent inhibition was observed at peptide concentrations between 16 and 75 μg/ml, with Pf1 (which includes both cysteines in Region II) showing the strongest inhibitive effect. The cysteines and the sulfhydryl groups were necessary for inhibition (Pf1A, Pf1B and Pf2A were not active). Pf70 which spans Region I and Pf25C had no inhibitory effect at concentrations as high as 250 μg/ml. The results are shown graphically in FIG. 7 wherein percent inhibition (after subtraction of background) is plotted as a function of peptide concentration. Pf1: open circles; Pf2: dark circles; Pf3: open upright triangles; Pf1A dark square; Pf1B open inverted triangles; Pf2A dark triangle; Pf70 open diamond; Pf25C dark diamond. Standard deviations were no greater than ±5% of the plotted mean.

### EXAMPLE 10

CS Binds to Microvilli of Hepatocytes in the Space of Disse

To identify the hepatocyte structures interacting with the CS, glutaraldehyde-fixed and LR White-embedded thin sections of rat liver were incubated with the recombinant CS polypeptide CS27IVC, mAb 2A10 and goat anti-mouse IgG-gold and analyzed by immunoelectron microscopy. Human liver tissue embedded in K4M was also labelled with CS27IVC followed by MAb 2A10 and protein A-gold.

Gold particles were found in areas of hepatocyte membrane exposed to the bloodstream, namely the microvilli protruding in the Space of Disse and to lateral membranes of adjacent hepatocyte up to the tight junctions that seal the bile canaliculi. Other regions of the hepatocyte plasma membrane were not stained, and neither were cell membranes of Kupffer cells or endothelia. Liver sections incubated with Falciparum-1 protein which lacks Region II+ or sections incubated only with monoclonal antibody 2A10 and/or with the gold conjugate were negative.

Intracellular hepatocyte labelling with CS27IVC was seen on the lysosomes in the vicinity of bile canaliculi possibly reflecting receptor internalization and degradation. The bile ducts themselves were negative. Lysosomal staining was also seen in Kupffer cells and, occasionally, in cells containing lipid droplets. On the whole, labelling was extremely localized.

Rat liver sections incubated with Falciparum-2 instead, showed essentially the same results but with lower staining intensity. The results were the same in rat mouse and human livers. Other tissues showed no specific labelling pattern. The results were repeated even after homogenization and fractionation (by density gradient centrifugation) of hepatocytes. The membrane fractions showed staining, but other fractions were not stained.

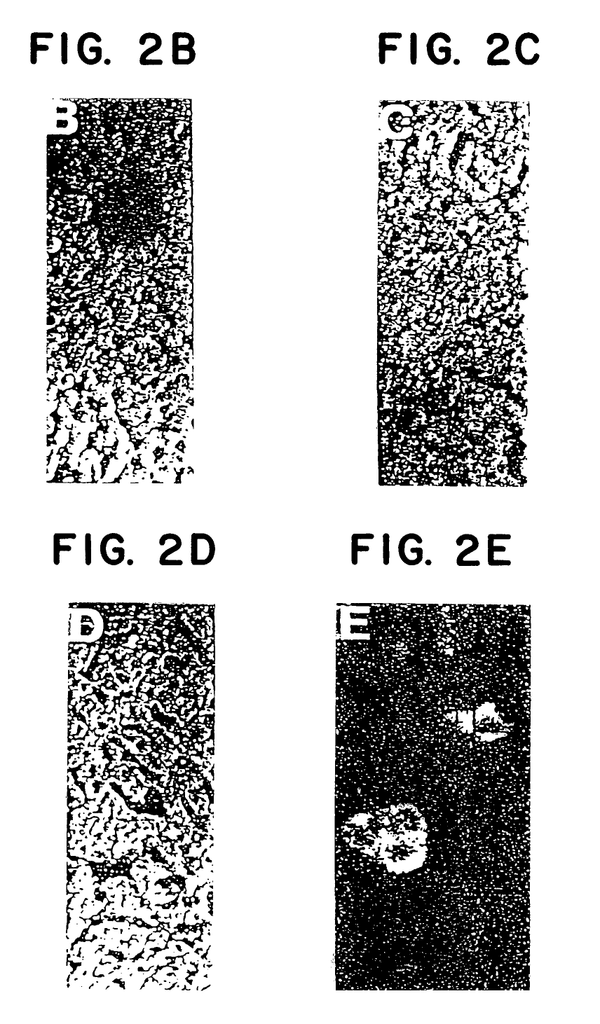

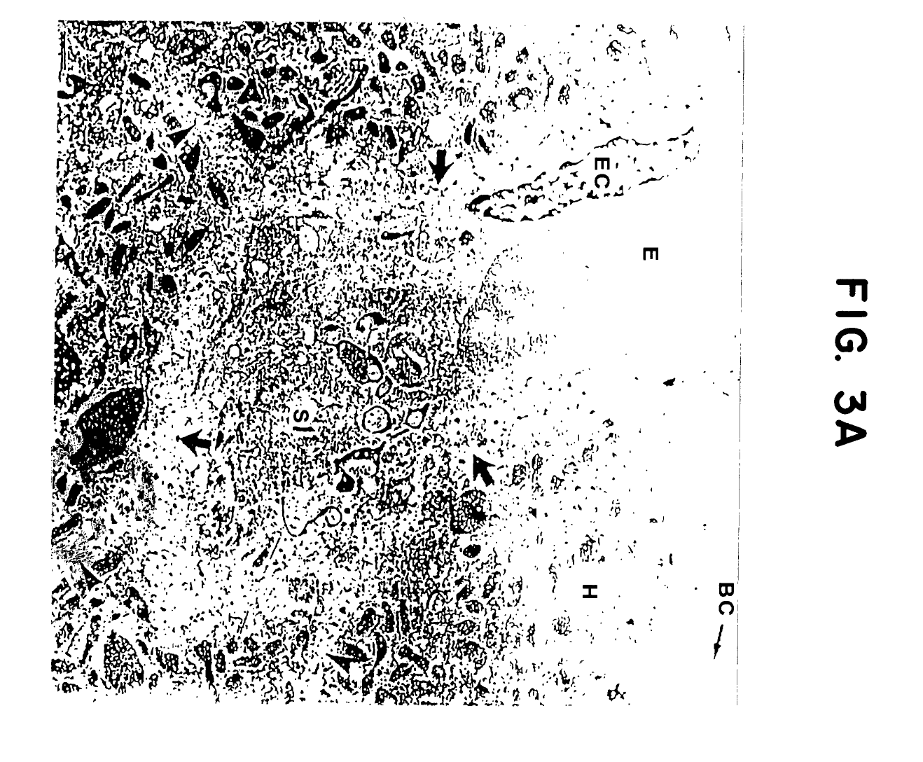

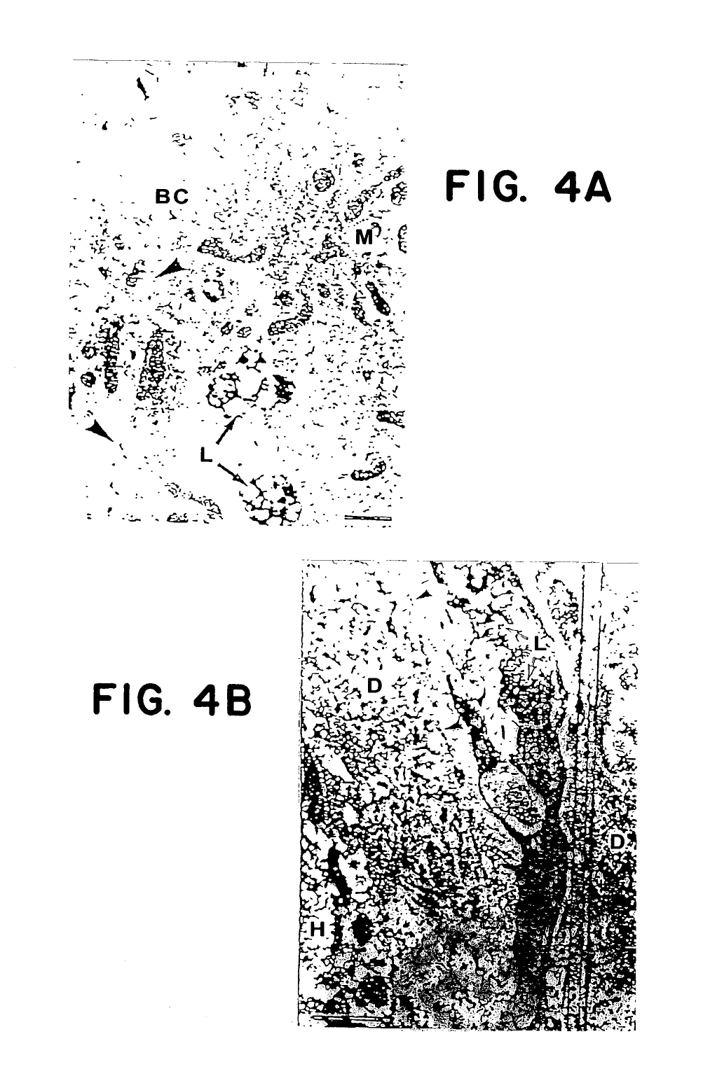

The results of some of these staining experiments are shown in FIGS. 3 and 4.

In FIG. 3A, it is apparent that the CS protein binds the entire hepatocyte surface except for the area exposed to the bile canaliculi; in FIG. 3B the binding of CS to the microvilli in the space of Disse is shown.

In FIG. 4A, it is apparent that the CS protein labels human lysosomes and also binds to the lateral hepatocyte cell membrane (arrowheads). (M stands for mitochondrium.)

In FIG. 4B, lysosomes (L) of Kupffer cells (K) are labelled but not the Kupffer cell surface nor the endothelial cell membrane (arrowheads) whereas the hepatocyte microvilli in the space of Disse are heavily labelled.

In FIG. 4C a rat liver cell membrane shows labelling of those membrane fragments that contain microvilli whereas other membranes (possibly bile canaliculi shown by arrowheads) are not labelled and contaminating cell organ cells (*) are not labelled.

In FIG. 4D, rat cell liver fractions containing mostly mitochondria (M) and rough endoplasmic reticulum (arrowheads) are not labelled.

### EXAMPLE 11

Aggregated, Region II+-Containing CS Binds to Liver Membranes

The gold conjugates employed in the foregoing liver section experiments contained no aggregates. However, the staining pattern was always patchy which suggested that the receptors were clustered, or the CS was aggregated, or both. Recombinant CS27IVC was subjected to molecular sieving as detailed above to isolate the monomeric form of the protein (which eluted between 66 and 200 kDa) from the aggregated form (which eluted at 669 kDa). The aggregated form of the recombinant polypeptide CS27IVC retained binding activity whereas the monomer form was inactive. Aggregates of Falciparum-1 protein were tested as a control and were found inactive, as expected. Falciparum-1 aggregates were formed by introduction of sulfhydryl groups followed by air oxidation.

The presence of aggregates or monomers in the active and inactive fractions, respectively, was confirmed by SDS-PAGE under reducing and nonreducing conditions followed by Western blotting using MAb 2A10. Reducing conditions resulted in the observation of a single band in all samples. Nonreducing conditions preserved several bands of increasing molecular weight in the FPLC fraction that had showed activity. (See, Example 5 above). The results are depicted in FIG. 5 which is a plot of OD v. FPLC fraction number. It shows that the CS protein eluted in two peaks, with the first one (fraction 9) corresponding to the aggregated form and the second one (fractions 12-14) corresponding mainly to monomeric forms.

### EXAMPLE 12

Peptide PbRII+ and Anti-RII+ Antibodies Inhibit Sporozoite Invasion of HepG2 Cells

HepG2 cells were incubated with P.berghei sporozoites in the presence of varying amounts of P.berghei peptide PbRII+ or control peptides. The number of exoerythrocytic forms (“EEF”) of the parasite that developed in the HepG2 cells were counted two days later. Because the viability and infectivity of sporozoites vary greatly, multiple experiments were performed so the results are statistically significant. HepG2 cells were plated at a density of 0.5×10⁶/ml, incubated for two hours with 50,000 P.berghei sporozoites per well in the presence of PbRII+ or other control peptides, or simply media, as indicated in Table 2 below. Cultures were grown for two days, fixed and stained with MAb 2E6 followed by goat anti-mouse IgG conjugated to horseradish peroxidase. In Table 2, “Number of EEF” represents the average number of schizonts counted per 20 fields under 20-times magnification of a light microscope in quadruplicate wells; P values were calculated by one-way analysis of variances corrected by the Bonferroni method using the commercially available computer program GraphPAD Instat, version 1.14, copyright 1990. In Experiment 6, peptides were added to the culture 2 hours after the addition of sporozoites, at which time invasion was complete.

TABLE 2
PbRII+ Inhibits P. berghei Sporozoite Invasion

| | Inhibitor | Concentration | Number of EEF^{a} | Percentage of | |
|---|---|---|---|---|---|
| Experiment | Peptide | (μg/ml) | (Mean ± SD) | Inhibition | P Value^{b} |
| 1 | PbRII+ | 250 | 53.7 ± 3.1 | 84.2 | <0.01 |
| | PbRII+ | 125 | 242.7 ± 23.8 | 28.5 | NS |
| | PCD59 (control) | 250 | 250.3 ± 103.2 | 26.3 | NS |
| | Medium alone | — | 339.7 ± 52.4 | — | |
| 2 | PbRII+ | 250 | 29.3 ± 8.6 | 66.8 | <0.01 |
| | Pf4 (control) | 250 | 122.5 ± 13.4 | 0.0 | NS |
| | Medium alone | — | 88.3 ± 9.3 | — | |
| 3 | PbRII+ | 250 | 35.7 ± 11.2 | 74.9 | <0.01 |
| | Pf4 (control) | 250 | 123.7 ± 20.1 | 13.2 | NS |
| | Medium alone | — | 142.5 ± 34.8 | — | |
| 4 | PbRII+ | 250 | 27.2 ± 4.8 | 81.5 | <0.001 |
| | Pf4 (control) | 250 | 105.0 ± 12.5 | 28.1 | NS |
| | Medium alone | — | 146.0 ± 28 | — | |
| 5 | PbRII+ | 500 | 0.0 | 100.0 | <0.001 |
| | Pf4 (control) | 500 | 375.2 ± 52 | 0.0 | NS |
| | Medium alone | — | 370.0 ± 47 | — | |
| 6^{c} | PbRII+ | 250 | 541.0 ± 186 | 0 | NS |
| | Medium alone | — | 523.5 ± 84 | 0 | |

HepG2 cells were plated at a density of 0.5 × 10⁶/ml, incubated for 2 hr. with P. berghei sporozoites (50,000 per well) in the presence of the PbRII+, negative control peptides (Pf4 and CD59), or media alone. Cultures were grown for 2 days, fixed, and stained with MAb 2E6, followed by goat anti-mouse immunoglobulin conjugated to horseradish peroxidase. Numbers in the fourth
column represent the average schizonts counted per 20 fields under 20 × magnification of a light microscope in quadruplicate wells (± the standard deviation).
^{a}EEF = exoerythrocytic forms.
^{b}P values were calculated using one-way analysis of variance (ANOVA), corrected by the Bonferroni method. NS, not significant.
^{c}In this experiment, peptides were added to the culture 2 hr. after the addition of the parasites, after completion of invasion.

As can be seen in Table 2 PbRII+ was effective in inhibiting EEF if it had been present during the initial phases of invasion. Two hours after addition of the sporozoites PbRII+ was no longer effective.

Several rabbits were hyperimmunized with PbRII+ conjugated to keyhole limpet hemocyanin. The antiserum titer of the animals was high (≧20,000 by ELISA). Nevertheless, only one antiserum reacted weakly with sporozoites (1:1000 by indirect immunofluorescence). The IgG fraction of this antiserum at 700 μg/ml significantly inhibited sporozoite invasion of HepG2 cells while preimmune IgG had no effect. Again the IgG fraction of immune sera was ineffective 2 hours after exposure of the cells to sporozoites. Thus, the ability of PbRII+ to inhibit binding of CS to the hepatocyte receptor was confirmed. The results of the IgG experiments are set forth in Table 3 below. Again, the 6th experiment involved delayed addition of the anti-PbRII+ antisera.

TABLE 3
Anti-PbRII+ Inhibits P. berghei Sporozoite Invasion

| | | Concentration | Number of EEF^{a} | Percentage of | |
|---|---|---|---|---|---|
| Experiment | Antibody | (mg/ml) | (Mean ± SD) | Inhibition | P Value^{b} |
| 1 | Anti-RII+ | 2.8 | 45.7 ± 22.6 | 84.4 | <0.01 |
| | Preimmune | 2.8 | 295.0 ± 68.8 | 0.0 | NS |
| | Medium alone | — | 293.5 ± 34.5 | — | |
| 2 | Anti-RII+ | 2.8 | 93.0 ± 76 | 70.6 | <0.01 |
| | Anti-RII+ | 1.4 | 111.3 ± 8.1 | 65.8 | <0.05 |
| | Anti-RII+ | 0.7 | 166.2 ± 38.4 | 47.4 | <0.05 |
| | Preimmune | 2.8 | 243.7 ± 38.9 | 23.1 | NS |
| | Medium alone | — | 316.0 ± 76.9 | — | |
| 3 | Anti-RII+ | 2.8 | 299.5 ± 26.1 | 46.1 | <0.01 |
| | Medium alone | — | 555.5 ± 7.7 | — | |
| 4 | Anti-RII+ | 2.8 | 187.3 ± 71 | 57.4 | <0.01 |
| | Preimmune | 2.8 | 347.5 ± 13.4 | 21.0 | NS |
| | Medium alone | — | 440.0 ± 59.9 | — | |
| 5^{c} | Anti-RII+ | 2.8 | 289.5 ± 25 | 16.5 | NS |
| | Preimmune | 2.8 | 344.0 ± 43.8 | 0.6 | NS |
| | Medium alone | — | 346.2 ± 13 | — | |
| 6^{c} | Anti-RII+ | 2.8 | 283.0 ± 45.3 | 27.2 | NS |
| | Preimmune | 2.8 | 316.2 ± 88.5 | 18.6 | NS |
| | Medium alone | — | 388.7 ± 41.4 | — | |

HepG2 cells were plated at 0.5 × 10⁶/ml, incubated for 2 hr with P. berghei sporozoites (50,000 per well) in the presence of protein A-purified anti-PbRII + IgG, preimmune IgG, or medium alone. Cultures were grown for 2 days, fixed, and stained with MAb 2E6, followed by goat anti-mouse immunoglobulin conjugated to horseradish peroxidase. Numbers in the fourth column represent the average
number of schizonts counted per 20 fields under 20 × magnification of a light microscope in quadruplicate wells (± the standard deviation).
^{a}EEF = exoerythrocytic forms.
^{b}P values were calculated using one-way analysis of variance (ANOVA), corrected by the Bonferroni method. NS, not significant.
^{c}In this experiment, peptides were added to the culture 2 hr. after the addition of the parasites, after completion of invasion.

Examples and Utility of Peptides According to the Invention

The foregoing experiments show that CS, in fact aggregated CS, recognizes specifically the basolateral domain of hepatocyte cell membrane. This specificity predicts the existence of a receptor on the hepatocyte cell surface. The ligand for this receptor (which is characterized below) resides within Region II+ of the CS protein, as defined by the present inventors.

Although the foregoing experiment focused on P. falciparum Region II+, the units are expected to be the same with Region II+ prepared from other species.

In fact, the peptide Pf1, an example of a peptide consisting essentially of Region II+, competes very effectively with recombinant CS and (at micromolar concentrations) with the P.berghei sporozoites. The peptide Pf2 which lacks the amino acids PCSVT competes much less effectively indicating that the missing sequence is part of the adhesion ligand (nevertheless, peptide Pf2 also binds to the same site as the CS protein). The cysteines in Region II+ are also important because analogs of Pf1 lacking only these cysteines were totally inactive.

Additional suitable peptides containing essential parts, or the entirety, of Region II+ can be easily identified using one or more of the above-described assays and the overlapping peptide method, which is a peptide screening technique well-known in the art and no more than routine experimentation. With reference to Table 1, peptides formed by omitting progressively one-by-one C-terminal amino acids from Regions II+ of different malarial species can be tested for CS-binding inhibitory activity. It has already been determined that the N-terminal and the positively charged amino acids of Region II+ are important.

Depending on the use for which they are intended, peptides and peptide-containing constructs within the present invention include the following:

(A) Peptides that inhibit the binding of CS protein (in this context “CS protein” includes recombinant CS polypeptides and entire sporozoites) by simply competing for receptor sites on hepatocytes. Such peptides should possess substantial CS-binding inhibitory activity (e.g., not substantially less than that of Pf2) and, if intended for use in vivo, should not elicit a substantial immune response from the host.

Such peptides may be as small as the minimum CS-binding inhibitory amino acid sequence from Region II+ or as large as CSFZ (Cys), i.e., consisting essentially of the CS-protein minus the immunodominant region. Such peptides should be soluble in aqueous media. Region II of Dame is excluded from the present invention.

The formation of disulfide-bond linked dimers, multimers, or aggregates of Pf1 (or CS) using the two cysteine residues probably imparts optimal binding activity, and the present inventors have evidence that Pf1 forms aggregates. Accordingly, peptides within the invention that compete for binding with CS should preferably be dimeric, multimeric, or aggregated. This is consistent with the finding that CS aggregates bind to hepatocytes and CS monomers do not.

Even though Region II+ is not itself immunogenic, immunogenic and non-immunogenic peptides incorporating it are nevertheless useful in inhibiting sporozoite invasion of hepatocytes. In fact, the absence of immunogenicity of Region II+ can be used to advantage in vivo, because the host to whom such peptides are being administered will not mount an immune response against them.

For example, Region II+-containing peptides or peptidomimetics can be administered to malaria susceptible subjects, for example intravenously, at sufficiently high concentrations to compete effectively with a subsequent challenge with sporozoites or to attenuate the severity of subsequent infection. These concentrations can be determined by means known to one of ordinary skill in the art. For example, optimum concentrations can be established using serially diluted preparations of the peptide in connection with a suitable testing procedure in rodents injected with P. berghei or P. yoelii. Preferred concentrations range from about 1 to about 10 mg in a mouse and from about 10 to about 100 mg in a human. Suitable vehicles for administration include, but are not limited to, isotonic saline. The peptides of the present invention can also be encapsulated in liposomes, the encapsulation having been described by Brenner, D., J.M.C.I. 78:1436, 1989; Anderson, P. et al., Cancer Research 50:1853, 1990; and Anderson, P. et al., J. Immunotherapy 12:19, 1992.

Peptides consisting of Region II+ or of the ligand adhesion portion thereof, are not expected to be toxic to the malaria-susceptible mammalian hosts because they were not toxic when administered to mice.

Such administration of Region II+ peptides would have to be preventive because they will have no effect on the blood stages or on the development of the liver stages of the parasite. Simultaneous infection and administration is about the limit of the CS-binding inhibitory effectiveness of Region II+ peptides.

(B) Alternatively, Region II+ peptides could be used to select monoclonal antibodies in vitro using the phage display technology (See, Barbas et al., Proc. Nat'l. Acad. Sci. U.S.A. 88:7978 (1991). These can be human or humanized monoclonal antibodies. Human origin or humanization would cause the immune system of the host to be “blind” to the antibodies. If administered to travelers, these antibodies to Region II+ would bind to the ligand adhesion site of the CS protein (or sporozoites) and thus prevent liver invasion through the hepatocyte receptor. Human chimeric and humanized antibodies of various predetermined specificities are engineered currently, See, e.g. Presta, L. G. Curr. Op. Struct. Biol. 2:593-596, 1992; and Burton, D. R., Hospital Practice, Aug. 15, 1992, 67-74 and references cited in each. See also Barbas, et al., Proc. Nat'l. Acad. Sci. USA 88:7978, 1991. The amount of monoclonal antibody administered should be sufficient to achieve a blood level ranging from about 1 to about 10 mg/ml. In vitro elicitation of antibodies can be performed according to methods known to those skilled in the art.

(C) The nonantigenic nature of Region II+ precludes the use of peptides containing Region II+ (and also containing an antigenic determinant) in vaccines. Nevertheless, peptides consisting essentially of the immunogenic amino acid sequences immediately following or preceding Region II+ of the CS protein can be used to immunize susceptible hosts. (See, Good et al., Annu. Rev. Immunol. 6:663-688, 1988). It seems likely that these antibodies would sterically hinder Region II+, and prevent infection.

In P.falciparum, such an antigenic C-proximal amino acid sequence is the sequence N K P K D Q L D Y Q N D I Q.

Other examples of such antigen sequences include, but are not limited to:

1. P S D K H I E Q Y L K K I K N S I (TH2R),
2. P S D Q H I E K Y L K R I Q N S L (TH2R), and
3. D K S K D Q L N Y A (TH3R).

See, e.g., Nussenzweig, V. and Nussenzweig, R., Sep. 15, 1990, Hospital Practice 45-57; Good, M. F., et al., Annu. Rev. Immunol. 6:563, 1988). Although polymorphic, TH2R and TH3R include only a few interspecies amino acid substitutions, and therefore, polymorphism would not be an impediment to incorporating them in a malaria vaccine preparation.

Structural and chemically functional mimetics of the peptides above are also within the scope of the present invention. Methods of preparation of such mimetics are described, for example, in Yamazaki et al., Chirality 3:268-276 (1991); Wiley et al., Peptidomimetics Derived From Natural Products, Medicinal Research Reviews, Vol. 13, No. 3, 327-384 (1993); Gurrath et al., Eur. J. Biochem 210:991-921 (1992); Yamazaki et al, Int. J. Peptide Protein Res. 37:364-381 (1991); Bach et al., Int. J. Peptide Protein Res. 38:314-323 (1991); Clark et al., J. Med. Chem. 32:2026-2038 (1989); Portoghese, J. Med. Chem. 34:(6) 1715-1720 (1991); Zhou et al., J. Immunol. 149 (5) 1763-1769 (Sep. 1, 1992); Holzman et al., J. Protein Chem. 10: (5) 553-563 (1991); Masler et al., Arch. Insect Biochem. and Physiol. 22:87-111 (1993); Saragovi et al., Biotechnology 10: (July 1992); Olmsteel et al., J. Med. Chem. 36:(1) 179-180 (1993); Malin et al. Peptides 14:47-51 (1993); and Kouns et al., Blood 80:(10) 2539-2537 (1992).

Such mimetics are typically non-peptide compositions that maintain the activity of the corresponding peptides because of structural and/or chemical functionality similarities. Among the advantages of mimetics are their relative lack of antigenicity and their ability to withstand degradation to which peptides are susceptible.

In addition, Region II+ peptides would be useful in drug design, i.e. in the construction of peptidomimetic molecules (for use in chemoprophylaxis) that bind to the CS hepatocyte receptor with sufficient affinity to inhibit the subsequent binding of sporozoites. An in vitro assay system for this purpose has been described above in Examples 4 and 8-12. It could employ for example HepG2 cells as targets and would test the ability of recombinant CS proteins to bind to their receptors in the presence or absence of a designed putative drug based on Region II+. The drugs which inhibit this binding would then be tested for their effectiveness in inhibiting sporozoite invasion of HepG2 cells, and in rodent malaria models (P. berghei and P. yoelii).

Peptides consisting essentially of Region II+, or its ligand adhesion subregions (see Table 1), can also be used for such drug screening and are more convenient for this purpose than recombinant CS-constructs. Such Region II+ derived peptides could be labelled and used exactly as the recombinant CS protein. The effectiveness of a drug would be accessed by its ability to inhibit the accumulation of the labelled Region II+ peptide in the liver of mice or other animals.

Furthermore, the peptides or mimetics above can be combined with a substance to be delivered to a hepatocyte. Such substances include, but are not limited to, DNA, such as genes, therapeutic agents such as drugs or other pharmaceutically active agents, or the like. The peptide or mimetic can be combined with the substance to form a complex through means known to those skilled in the art such as substitution, insertion, or conjugation with the peptide or mimetic. See, Mulligan, Science 260:926-932 (May 14, 1993); A. D. Miller, Hum. Gene Ther. 1:5 (1990): N. Jones and T. Shenk, Cell 16:683 (1979); K. L. Berkner, BioTechniques 6:616 (1988); F. L. Graham and L. Prevea, in Methods in Molecular Biology 7:109-127, E. J. Murray, Ed. (Humana, Clifton, N.J., 1991); H. A. Jaffe et al., Nat. Genet. 1:374 (1992); X. O. Breakefield and N. A. DeLuca, New Biol. 3:230 (1992); G. Y. Wu, J. Biol. Chem. 266:14338 (1991); R. J. Christiano et al., ibid 90:2122 (1993); D. M. Bodine et al., Exp. Hematol. 19:206 (1991); T. Ohashi et al., Proc. Natl. Acad. Sci. U.S.A. 89:11332 (1992) P. H. Correll et al., Blood 80:331 (1992); J. M. Wilson et al., J. Biol. Chem. 267:963 (1992); J. M. Wilson et al., ibid, p. 22483; M. Kaleko et al., Hum. Gene Ther. 2:27 (1991); N. Ferry et al., Proc. Natl. Acad. Sci. U.S.A. 88:8377 (1991); J. B. Weinberg et al., J. Exp. Med. 174:1477 (1991); P. Lewis et al., EMBO J. 11:3053 (1992); B. Sauer and N. Henderson, Proc. Natl. Acad. Sci. U.S.A. 85:5166 (1988); A. Helenius, Cell 69:577 (1992). For example, the peptide or mimetic can be incorporated into the envelope protein of a recombinant virus to enhance or achieve the capture of the recombinant virus by the hepatocyte.

These complexes will deliver or target the substance to the hepatocyte when administered to a mammal. The substance will be delivered in a single or a cumulative therapeutically effective amount which can be determined by means known to those skilled in the art such as by developing a matrix and assigning a dosage to each point in the matrix.

Administration of these complexes can be by any manner known to those skilled in the art, including but not limited to, oral or parenteral administration.

The Hepatocyte Membrane Receptor for CS Protein

The existence of a receptor for the CS protein in the basolateral domain of hepatocyte plasma membranes has been shown in the experiments by the localization and specificity of the binding and the resemblance of the foregoing experimental results to other receptor-ligand interactions involving different proteins and their receptors.

CS receptors or the basolateral domain of the plasma membrane of hepatocytes have been purified and identified as heparan sulfate proteoglycans (HSPG) of 400-700,000 Mr, which are tightly associated with the cell membrane. This characterization is based on observations made at the tissue, cellular, and molecular levels.

Additionally, it has been determined that CS protein binds to the receptor via the particular motif CSVTCGXXXXXRXR. Previous work had suggested that the receptor was a sulfated glycoconjugate because the same motif, CSVTCGXXXXXRXR, determined the binding of the CS to sulfatide and cholesterol-3-sulfate.

The following materials and methods were used in Examples 13-20 below.

Materials

For the co-immunoprecipitation studies, either rat (Brown Norway) liver, kidney tissue, or HepG2 cells (ATCC HB8065—Rockville, Md.) metabolically labeled with carrier-free Na₂³⁵SO₄ (Amersham—Arlington, Ill.) or with ³⁵S-methionine and cysteine (Tran³⁵S-label; ICN—Costa Mesa, Calif.) were used. As ligands for the putative receptors, four recombinant CS proteins (Cerami et al., Cell 70:1021-1033, 1992) were used. Two of these were E. coli-derived (Hoffmann-LaRoche): CS27IVC, which contains Region I, Region II+ and a full representation of the repeat domain as is described above; CSFZ(Cys), which is identical to CS27IVC, except that it contained only a single copy of the repetitive sequence NANP and is described above. The other two recombinants, Falc-1 and Falc-2, which also are described above, were yeast-derived and were obtained from the Chiron Corporation Corporation, Emeryville, Calif. Both Falc-1 and Falc-2 contain Region I and the repeat domain, but only Falc-2 contains Region II+. The HepG2 binding assays were performed in Removawell tissue culture plates (Dynatech Laboratories—Chantilly, Va.). For the immunoelectron microscopy studies, glutaraldehyde (grade I—Sigma—St. Louis, Mo.) and paraformaldehyde (Kodak—Rochester, N.Y.); Lowicryl K4M and LR White (Polysciences—Warrington, Pa.) and protein A gold 15 nm (PAG15) (Amersham) were used. For proteoglycan purification, G-25 columns (PD-10; Pharmacia—Piscataway, N.J.) and Centricon-10 units (Amicon—Beverly, Mass.) were used. Other reagents were Ham's F-12 medium (Sigma)—St. Louis, Mo.); methionine-free Dulbecco's Modified Eagle Medium (D-MEM; Gibco—Grand Island, N.Y.); the monoclonal antibody 2A10 (Nardin et al., J. Exp. Med. 156:20-30, 1982) detecting the repeat domain of P. falciparum CS; prefixed Staphylococcus aureus cells (Staph A cells; Pansorbin, Calbiochem—La Jolla, Calif.); heparinase II (Sigma) and heparitinase (ICN), chondroitinase ABC (Boehringer Mannheim, Indianapolis, Ind.; ICN), pronase (Boehringer), heparin and chondroitin sulfate (both Sigma); phosphatidylinositol-specific phospholipase C (PI-PLC) (Boehringer).

Immunoelectron Microscopy

Normal rat kidney tissue was fixed with 4% paraformaldehyde and 1% glutaraldehyde in phosphate-buffered saline (PBS) and embedded in LR White; normal human liver was fixed similarly and embedded in Lowicryl K4M (Cerami et al., Cell 70:1021-1033, 1992). Ultrathin sections were sequentially labeled with 10-50 μg/ml CS27IVC, 15 μg/ml mAb 2A10 and a 1:30 dilution of PAG15. Prior to immunolabeling, part of the sections were incubated for 120 min at 37° C. with heparitinase and chondroitinase ABC (both ICN) using concentrations of 20 U/ml and 2 U/ml, respectively. Control specimens were incubated only with the gold markers or with mAb 2A10 and PAG15.

Biosynthetic Labeling

Rats received two intraperitoneal injections of 1 mCi carrier-free Na₂³⁵SO₄, 24 and 12 hr before they were euthanized. The livers and kidneys were chilled on ice, minced, and homogenized in lysis buffer A (1% Triton X-100, 100 mM NaCl, 50 mM Tris-HCl, pH 7.2, 1% bovine serum albumin (BSA) (Sigma) containing 1 mM PMSF, 5 μg/ml of each leupeptin, pepstatin and antipain (all Boehringer) as protease inhibitors). After shaking for 2 hours, the lysates were centrifuged at 16,000 g for 30 minutes. The supernatants were then used for co-immunoprecipitation with the CS recombinant proteins.

Semi-confluent HepG2 cell monolayers were incubated with 100 μCi/well Na₂³⁵SO₄ in Ham's F-12 medium supplemented with 1-2% fetal calf serum (FCS) equilibrated with PBS by Sephadex G-25 passage. The incubation was performed in 6 well plates (Costar, Cambridge, Mass.) for 6 hr. After washing with PBS, the cells were scraped off the plate, and treated with lysis buffer A for 30 min on ice. Supernatants for co-immunoprecipitation were obtained by spinning the lysates for 30 minutes at 16,000 g.

A separate sample of the cells was metabolically labeled with 100 μCi/well ³⁵S-methionine/cysteine for 3 hours in methionine-free MEM containing 1% FCS equilibrated in PBS by Sephadex G-25 passage.

Release of Proteoglycans From the Surface of HevG2 Cells

Adherent HepG2 cells labeled with Na₂³⁵SO₄ as described above were washed 3× with cold PBS, and then incubated for 5 minutes at 4° C. with PBS containing 0, 2.5, 5, 10, 20 or 40 μg/ml trypsin. The supernatants were removed, and 200 μg/ml soybean trypsin inhibitor (Sigma) was added. The trypsin-treated cells were then scraped off in PBS/BSA, and extracts were prepared in lysis buffer A as above.

Separate samples of HepG2 cells labeled with sulfate were treated with 0, 31.25, 62.5, 125, 250 or 500 μg/ml heparin in medium for 10 min at 25° C., or incubated with 0, 6.25, 12.5, 25, 50 and 100 U/ml of PI-PLC for 50 min at 37° C. The supernatants of the treated cells were analyzed by SDS-PAGE and by co-precipitation with the CS. Prior to co-immunoprecipitation with the recombinant CS proteins, the supernatants were mixed with 1/5 volume of lysis buffer A. The cell pellets were scraped off and were lysed with buffer A as above.

CS/proteoglycan Co-immunoprecipitation

All extracts and supernatants were first precleared with normal rabbit serum and Staph A cells. Then 32 μg/ml of CSFZ(Cys) or 64 μg/ml of CS27IVC, Falc-2 or Falc-1 were added for 30 minutes, followed by 10.8 μg/ml mAb 2A10 for 30 minutes, and 1% Staph A cells (final concentrations). The suspensions were shaken for 1 to 5 hours at 4° C. The Staph A cells were washed 3 times with the lysis buffer A, 2 times with lysis buffer B (10 mM Tris-HCl, pH 7.2, containing 100 mM NaCl and 0.5% NP40), 1 time with 50 mM Tris-HCl, ph 7.2, and were processed in this buffer for electrophoresis under reducing and non-reducing conditions.

Samples of the immunoprecipitates were incubated with 0.1 U/ml heparitinase (ICN) in 50 mM Tris-HCl, pH 7.0, containing 0.1% BSA; or with 1 U/ml chondroitinase ABC (ICN) in 50 mM Tris-HCl, ph 8.0, containing 0.1% BSA and 50 mM sodium acetate for 3 hr at 37° C. in the presence of protease inhibitors; or with 100 μg/ml pronase for 1 hour at 37° C. After this treatment, the Staph A cells were washed and processed as above.

SDS-PAGE

³⁵SO₄-labeled immunoprecipitates were examined on either 5% or 3 to 20% gradient polyacrylamide gels (Laemmli, Nature 128:2009-2012, 1970). The gels were fixed with 10% glacial acetic acid and 30% methanol, impregnated for 30 min in 1 M salicylic acid, dried and exposed to Kodak X-Omat AR film at −70° C.

Purification of HepG2 Cell Proteoglycans

Trypsin-released and ³⁵SO₄-labeled proteoglycans were equilibrated in 50 mM Tris-HCl, pH 6.5 by passage in a Sephadex G-25 column, and concentrated by Centricon-10 centrifugation. Urea (Boehringer) was added to a final concentration of 7 M, and the sample subjected to anion exchange chromatography in a mono-Q column (Pharmacia) using a Pharmacia FPLC apparatus. Elution was performed with 50 ml of a 0 to 2 M NaCl gradient in 50 mM Tris-HCl, ph 6.5, containing 7 M urea. One ml fractions were collected and radioactivity counted in 20 μl aliquots. Positive fractions were pooled, equilibrated in 50 mM Tris-HCl, pH 7.2, and 7 M urea and concentrated as above. A 200 μl sample (22,500 cpm) was subjected to a Superose 6 column and eluted with 50 mM Tris-HCl, pH 6.5, and 7 M urea. Fractions (0.5 ml) were collected, and the cpm were counted in 20 μl aliquots. Thyroglobulin (669 kDa), apoferritin (443 kDa), β-amylase (200 kDa) and alcohol dehydrogenase (150 kDa) were used as molecular weight markers (all Sigma). The combined positive fractions from 3 column runs were pooled and were concentrated to a final volume of 1 ml. Part of this preparation was subjected to hydrolysis and amino acid and amino sugar analysis. For amino acid analysis the hydrolysis was with 6N HCl at 110° C. for 22 hours, and for amino sugars with 4N HCl at 110° C. for 7 hours. Analysis was performed using Waters Maxima software, 510 pump, and 490 detector. A Waters Novapak C8, 15 centimeter column was used.

Inhibition of binding of CS to HepG2 cells

10⁵ HepG2 cells were deposited in 96-well Removawell tissue culture plates and allowed to grow overnight in MEM (Gibco) containing 10% FCS, 1 mM L-glutamine (Gibco), 3 mg/ml glucose (Sigma) and 1× non-essential amino acids (Gibco). The HepG2 cells were fixed with 4% paraformaldehyde in TBS (50 mM Tris-HCl, pH 7.5, 137 mM NaCl, 2 mM KCl), were washed 3 times with TBS, and were stored at 4° C. in BSA/TBS until use. CSFZ(Cys) at 5 μg/ml was incubated with increasing amounts of the presumed inhibitors at 37° C. for 15 min. Fifty μl of these mixtures were added to the cells, incubated for 1 hr at 37° C., washed 3× with TBS/0.05% Tween and then sequentially incubated with 50 μl of mAb 2A10 at a concentration of 10 μg/ml in TBS/BSA buffer for 30 min at 37° C., and 1:5000 dilution of goat anti-mouse IgG conjugated to alkaline phosphatase (Boehringer) for 30 minutes. Bound enzyme was revealed by the addition of the fluorescent substrate, 4-methylumbelliferyl-phosphate (Sigma) in 100 mM Tris-HCl, pH 9.5, 100 mM NaCl and 5 mM MgCl₂. After 15 minutes, the fluorescence was measured in a Fluoroskan II plate reader (ICN).

In some experiments the HepG2 cells were treated with either heparitinase (Sigma) or chondroitinase ABC (ICN) before incubation with the CS. Heparitinase treatment was performed in 0.05 M acetate buffer, pH 6.0, containing 1 mg/ml BSA and 1 mM PMSF, 5μg/ml leupeptin and pepstatin. Cells were treated for 3 hours at 37° C. and then were washed 3× with TBS before protein was added. Chondroitinase ABC treatment was performed overnight at 37° C. in 0.1 M Tris/HCl, pH 8.0, 0.03 M sodium acetate and 0.1% BSA, and washed 3× with TBS. Enzyme-treated cells were then incubated with CS as outlined above.

### EXAMPLE 13

Heparitinase Digestion of the Plasma Membrane Receptors for CS

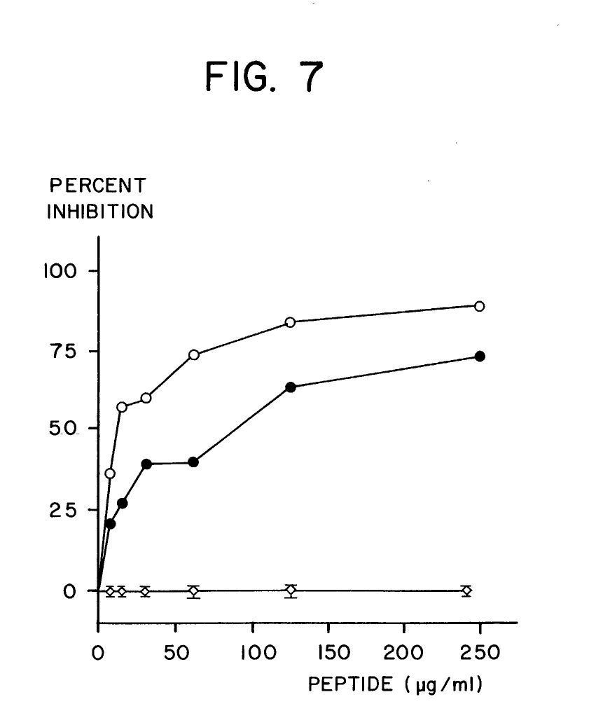

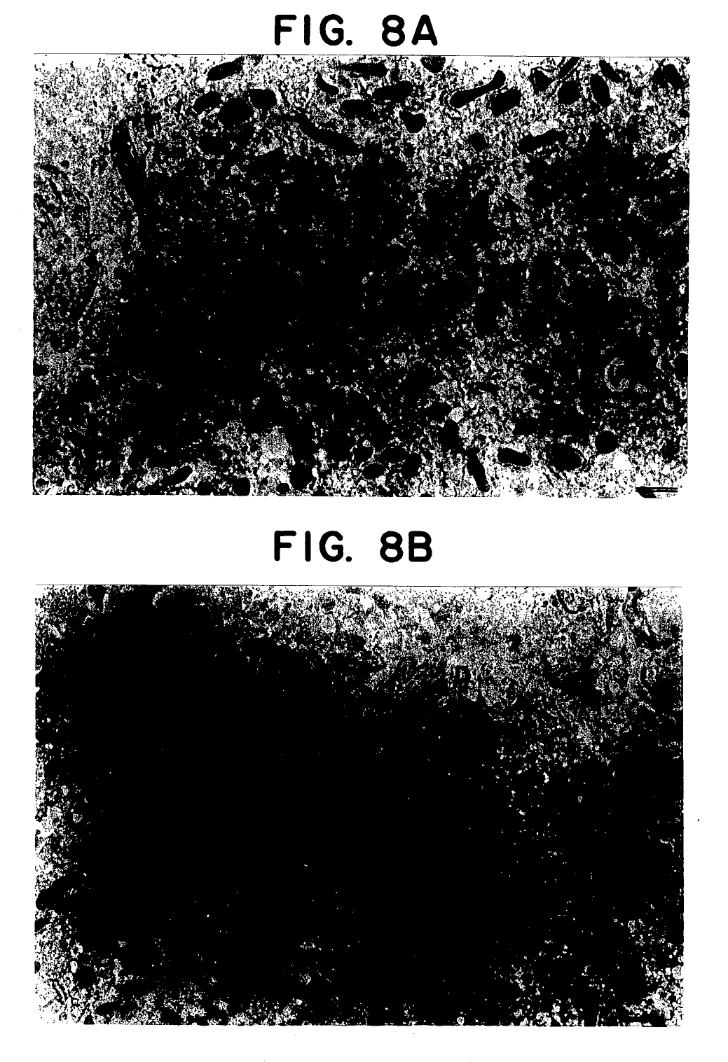

Human liver sections were incubated with heparitinase or chondroitinase ABC for 2 hours at 37° C., prior to immunolabeling with recombinant CS. The sections were then labelled with CS27IVC followed by mAb 2A10 and PAG15. FIGS. 8a and 8b are electron micrographs of Lowicryl K4M-embedded sections. The section of FIG. 8a was treated with 2 U/ml chondroitinase ABC (ICN) for 2 hour at 37° before the immunolabeling. The typical CS label on the space of Disse (D) and on the lateral domain of the hepatocyte plasma membrane (arrows) and on lysosomes (L) was unaltered. The patchy pattern of CS-labeled hepatocyte microvilli within the space of Disse was still present in the chondroitinase-treated sections (FIG. 8a). The section of FIG. 8b was treated with 20 U/ml heparitinase (ICN) under identical conditions, and subsequent CS staining was not observed in the space of Disse (D). Only a faint label was left on the lysosomes (L).

### EXAMPLE 14

Inhibition of CS Binding to HepG2 Cells

Proteoglycans were preincubated with 5 μg/ml of CSFZ(Cys) and then were added to paraformaldehyde-fixed HepG2 cells. Binding of CS to the HepG2 cells was revealed by mAb 2A10, followed by anti-mouse alkaline phosphatase-conjugated IgG. Bound enzyme was revealed by a fluorescent substrate, 4-methylumbelliferyl-phosphate. Each point represents the mean of triplicates. Percent inhibition was calculated by comparison to CSFZ(Cys) preincubated in medium alone.

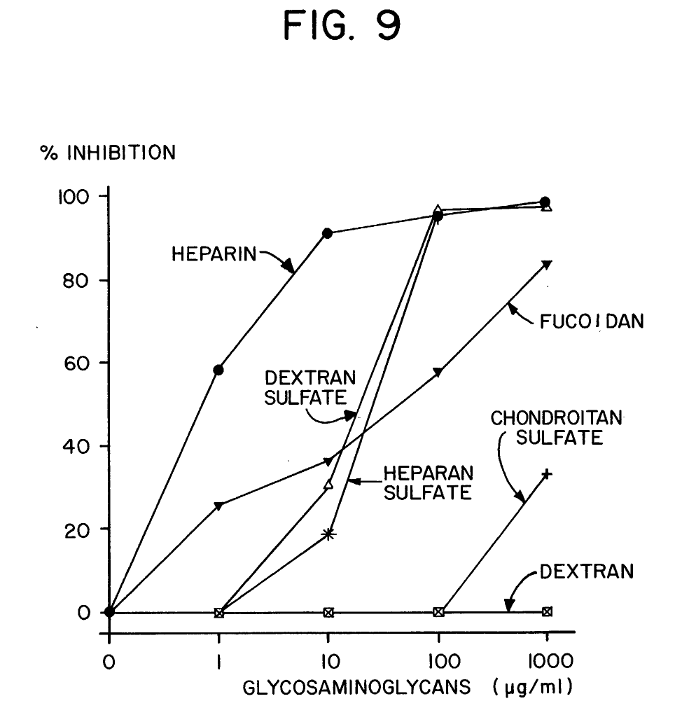

FIG. 9 illustrates that the CS binding to HepG2 cells was inhibited by heparin, heparan-sulfate, dextran-sulfate and fucoidan, but not by chondroitin sulfate and dextran.

### EXAMPLE 15

Inhibition of CS Binding to HepG2 Cells

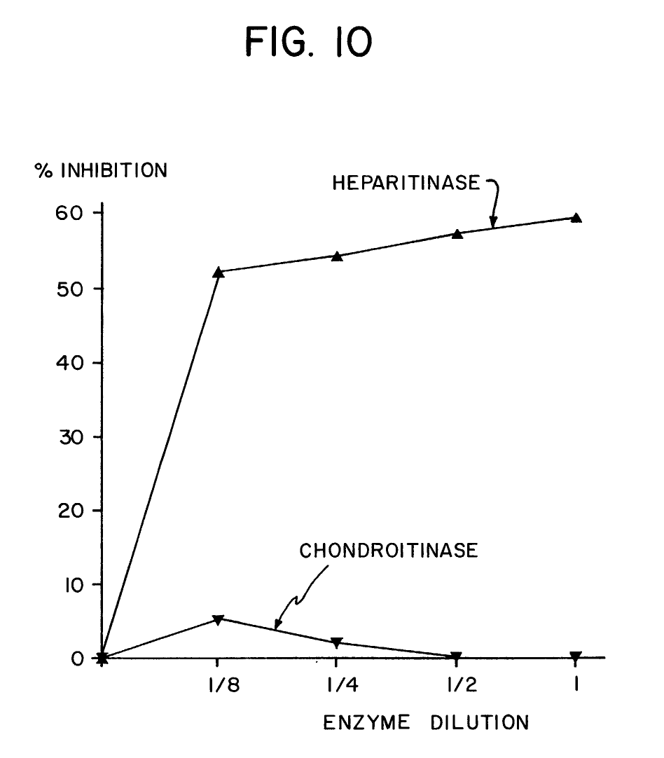

Paraformaldehyde-fixed HepG2 cells were preincubated with varying amounts of enzyme, washed and then incubated with 5 μg/ml of CSFZ(Cys). CS binding was revealed as described in Example 14. Heparitinase and chondroitinase ABC were used at initial concentrations of 5 U/ml and 1 U/ml, respectively. As shown in FIG. 10, heparitinase treatment prevented CS binding, while chondroitinase ABC treatment had no effect. Each point represents the mean of triplicates, and percent inhibition was calculated by comparison to cells that were not treated with enzyme.

### EXAMPLE 16

Binding of CS to Kidney Tubular Basement Membrane and to Tubular Epithelia

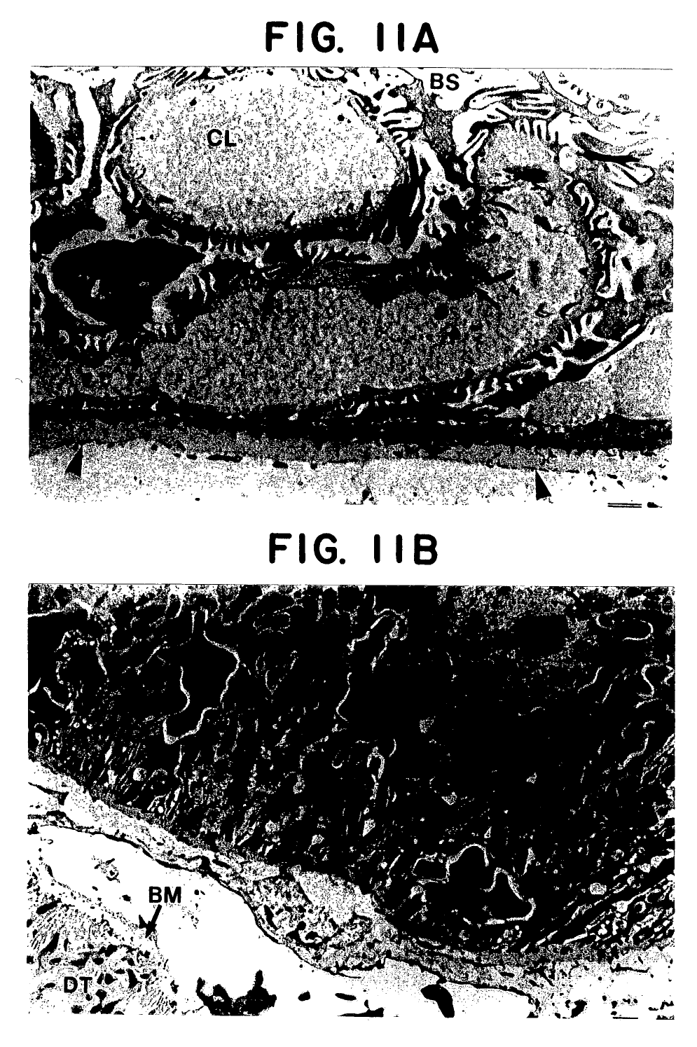

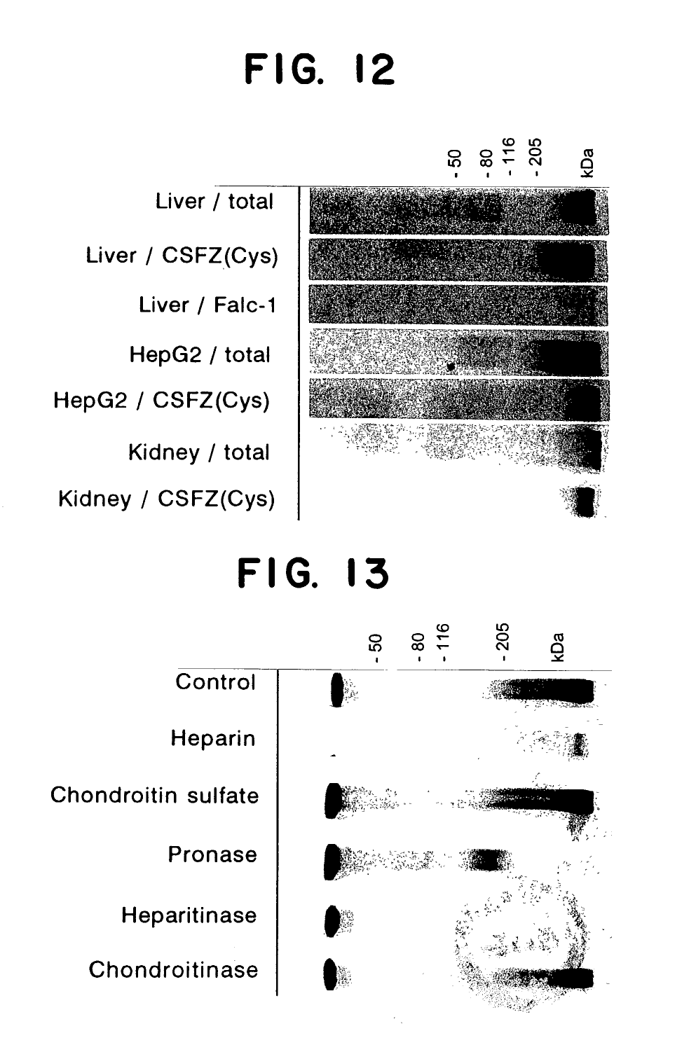

Electron micrographs of LR White-embedded rat kidney sections labeled with CS27IVC, mAb 2A10, and PAG15 were studied. A highly selective CS staining in the rat kidney in a pattern consistent with proteoglycan involvement was detected. FIG. 11a illustrates that in the glomerulus, the CS binding was restricted to the basement membrane of the Bowman capsule (BC) and predominantly to the laminae rarae (arrowheads), as well as to the proximal tubules, Henle loop, distal tubules, and collecting tubules. The glomerular basement membrane below the fenestrated capillary endothelium (arrows) was unstained; CS did not bind there.

FIG. 11b illustrates that the epithelia of the distal tubule (DT) show CS label only on the laminae rarae of the basement membrane (BM). In contrast, the epithelia of the proximal tubule (PT) are also stained on their basolateral domains and lysosomes (L).

The apical microvilli and all the other kidney epithelia were negative. The proximal epithelia showed intracellular CS staining of the lysosomes.

To verify that proteoglycans were involved in CS binding, the kidney sections were treated either with heparitinase or with chondroitinase ABC prior to CS staining as in Example 15. Again, only heparitinase abolished the staining.

### EXAMPLE 17

CS Binding to Heparan-Sulfate Proteoglycans

Extracts of cells metabolically labeled with ³⁵SO₄ were incubated for 30 minutes at 4° C. with 32 μg/ml of recombinant CSFZ(Cys) or 64 μg/ml Falc-1, and were immunoprecipitated with mAb 2A10 and Staph A cells. From the total liver lysate, CSFZ(Cys) co-immunoprecipitated sulfated molecules which migrated as a smear on top of the SDS-PAGE. Immunoprecipitation with Falc-1 was negative. The SDS-PAGE patterns of the total lysate of HepG2 cells, and of the molecules co-immunoprecipitated with CSFZ(Cys) resembled the corresponding liver SDS-PAGE patterns. In kidney lysates, CSFZ(Cys) selectively immunoprecipitated a band of slightly lower molecular weight than the total kidney sulfated molecules.

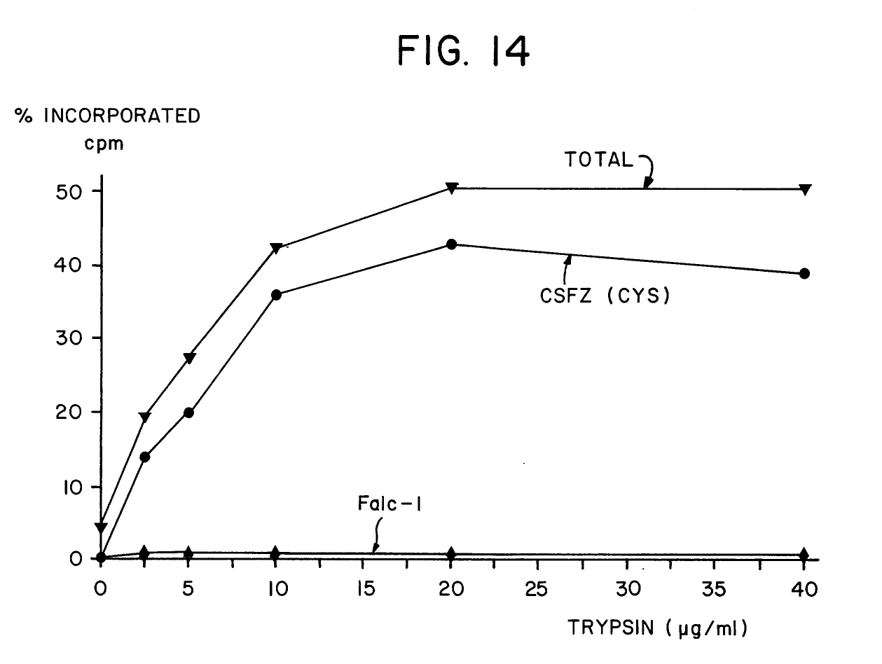

FIG. 12 demonstrates that the recombinant protein CSFZ(Cys) bound to sulfated macromolecules migrating as a smear on top of the SDS-PAGE gel, in a pattern typical of proteoglycans. Identical results were obtained when two other CS recombinants containing Region II−+ (CS27IVC and Falc-2) were used, except that the immunoprecipitation was less efficient. Negative results were obtained with recombinant Falc-1, which does not contain Region II-+.

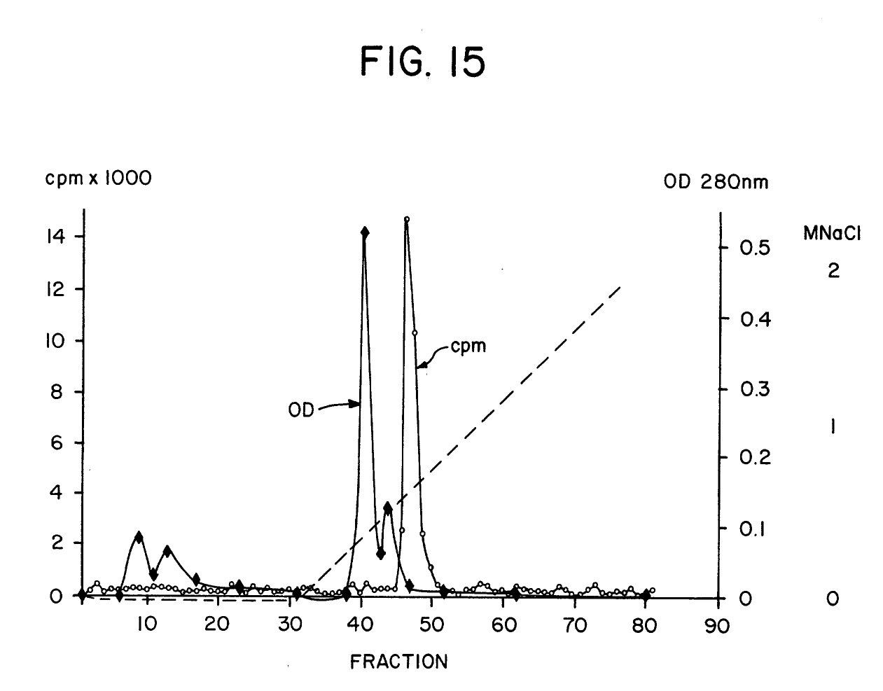

Several additional observations indicate that the sulfated macromolecules which bind CS are heparan sulfate proteoglycans. The high Mr bands on SDS-PAGE disappeared after treatment of the immunoprecipitated material with 0.1 U/ml heparitinase for 3 hours at 37° C. This led to complete degradation of the high molecular weight smear, whereas 1 U/ml chondroitinase ABC had no effect as illustrated in FIG. 13. Digestion of the immunoprecipitates with 100 μg/ml pronase for 1 hour at 37° C. after co-immunoprecipitation, resulted in a shift of the labeled smear to 200 kDa and in a significant decrease of the apparent molecular weight of the sulfated molecules, which migrated as a broad 200,000 Mr band in SDS-PAGE. (See FIG. 13) Also, co-immunoprecipitation of the CS receptor by CSFZ(Cys) was prevented by previous addition of 100 μg/ml heparin to ³⁵SO₄-labeled HepG2 cell extracts (control lane), whereas the same concentration of chondroitin sulfate had no effect. (See FIG. 13)

### EXAMPLE 18

The Nature of Membrane Attachment of the CS Cellular Receptors

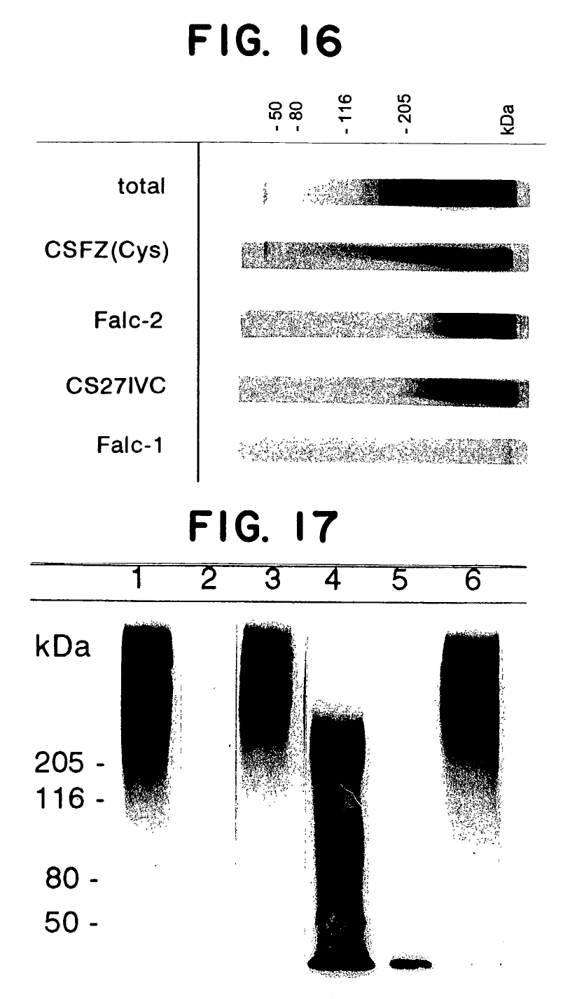

Sub-confluent HepG2 cells were incubated with various concentrations of trypsin for 5 minutes at 4° C. Sulfate label was released in the supernatant in a dose-dependent manner, and maximum release (50% of the total incorporated cpm) was reached at 20 μg/ml trypsin as illustrated in FIG. 14. Independently of the trypsin concentration, about 80% of the released HSPG was co-immunoprecipitated by 32 μg/ml CSFZ(Cys) and 10.8 μg/ml mAb 2A10. Under the same conditions, Falc-1 was negative.

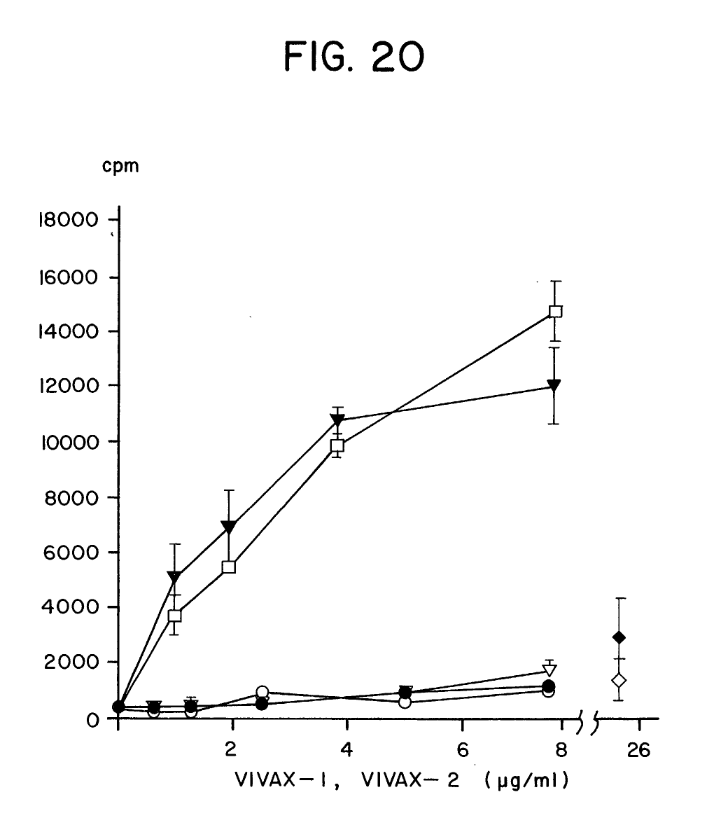

This indicates that a large proportion of the newly synthesized membrane-associated HSPG binds CS. From the total HepG2 cell extracts prior to trypsin treatment, and from the cell extracts after trypsin treatment, 54% and 37% of the counts, respectively, were co-immunoprecipitated. The trypsin-insensitive, immunoprecipitable counts most likely represented HSPGs which are either intracellular or are associated with the membrane in areas of cell attachment and, therefore, are inaccessible to the enzyme. On SDS-PAGE gels, the cell-associated, and the trypsin-released molecules ran similarly as high molecular weight smears on top of the gel, indicating that mild trypsinization of the HepG2 cells does not lead to extensive degradation of the proteoglycan core proteins (compare patterns in FIG. 13 and in insert of FIG. 17).

To analyze further the mode of association of the CS receptor to the membrane of the ³⁵SO₄-labeled HepG2 cells, the cells were treated with 0-100 U/ml phosphatidylinositol-specific phospholipase C (PI-PLC) for 60 minutes at 37° C. or with 0-500 μg/ml heparin for 10 minutes at 25° C., respectively. Neither treatment lead to the specific release of sulfate-labeled molecules.

In an attempt to identify the core protein of the malarial protein HSPG receptor, HepG2 cells were metabolically labeled with ³⁵S-methionine and ³⁵S-cysteine for 3 hours. As revealed by SDS-PAGE, the HepG2 cell extracts contained a very large number of radiolabeled proteins. However, following mild trypsinization as above (20 μg/ml, 5 minutes 4° C.), no counts were released in the supernatant above background.

### EXAMPLE 19

Binding of Different Constructs to the Receptor

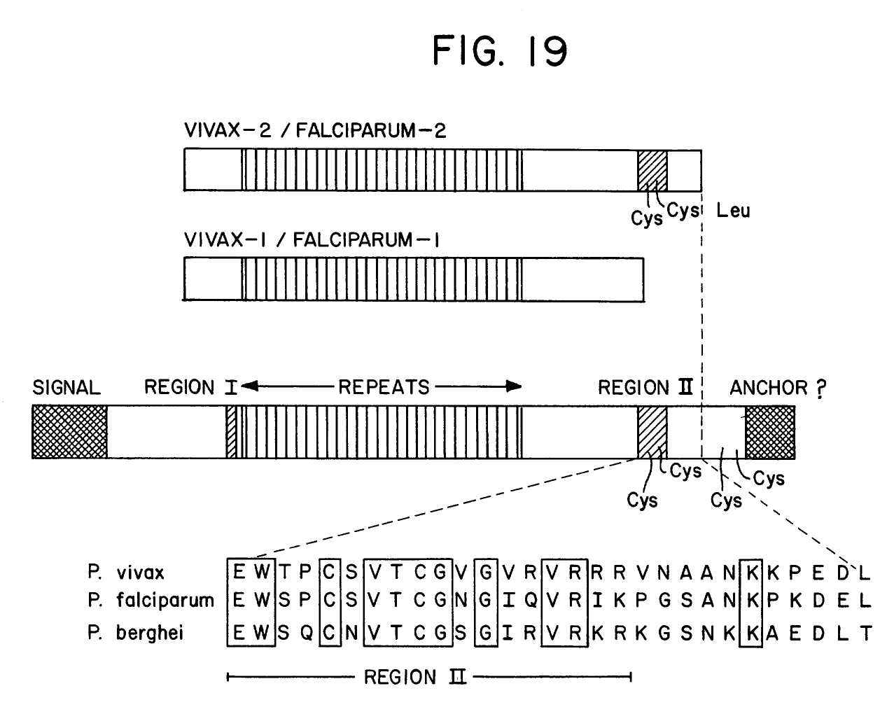

³⁵SO₄-labeled trypsin-released proteoglycan (total supernatant) was precipitated by CSFZ(Cys) (32 μg/ml), Falc-2 (64 μg/ml), CS27IVC (64 μg/ml) and Falc-1 (64 μg/ml). The labeled molecules were incubated with CSFZ(Cys), CS27IVC, and Falc-2 at equivalent concentrations of Region II+, and co-immunoprecipitated with the mAb 2A10. CSFZ(Cys), lacking most of the repeat region, precipitated up to 86% of the total sulfate-labeled proteoglycans (see FIG. 14), while CS27IVC and Falc-2 precipitated only 18% and 6% of the counts, respectively. Falc-1, which lacks Region II+, was negative. Nevertheless, the molecules precipitated by CS27IVC, CSFZ(Cys), and Falc-2 migrated identically on SDS-PAGE as illustrated in FIG. 16, and were equally susceptible to heparitinase and pronase digestion. The high molecular weight of the immunoprecipitated material shows that trypsin did not cause extensive degradation of the CS receptors.

Because aggregation of the recombinant CS is a prerequisite for receptor recognition, the observed differences may reflect the degree and/or type of aggregate formation in the various Region II+ containing constructs. Analysis of the aggregates was complicated by the fact that the aggregates are formed by covalent (disulfide bonds) and non-covalent interactions (Cerami et al., Cell 70:1021-1033, 1992.

### EXAMPLE 20

Purification of the CS Receptor from HepG2 Cell Membranes

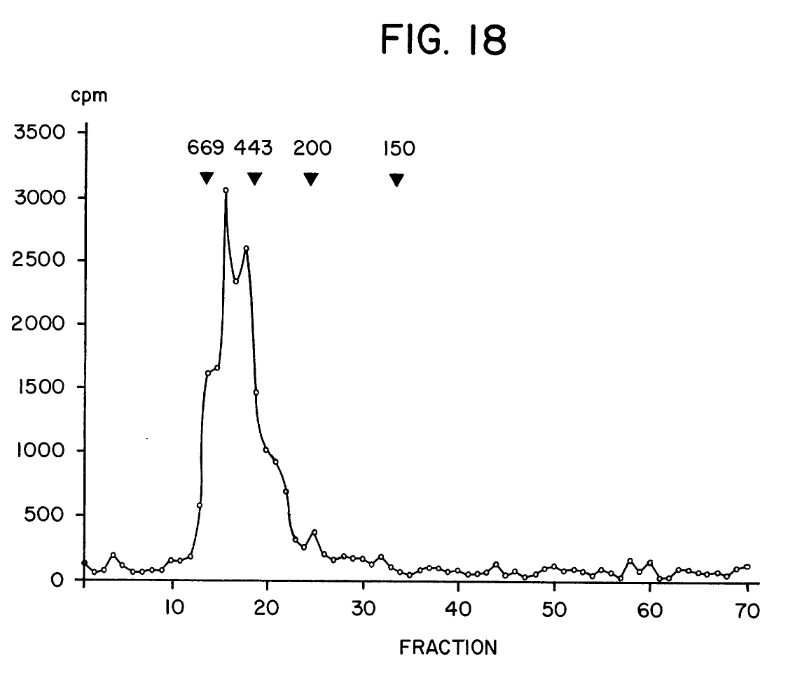

Subconfluent sulfate-labeled HepG2 cells were incubated with 20 μg/ml trypsin for 5 minutes at 4° C., and the supernatants were loaded onto an anion exchange chromatography Pharmacia mono-Q column. These were eluted with a 0 to 2 M NaCl gradient in the presence of 7 M urea. The radiolabelled proteoglycan was eluted from the column as a sharp peak at a NaCl concentration of 0.6 to 0.8 M as illustrated in FIG. 15, and about 80% of the counts in this peak were co-immunoprecipitated by CSFZ(Cys). Protein was not detectable in the labeled peaks. One ml fractions were collected and counted.

FIG. 17 illustrates that heparin, but not chondroitin sulfate, inhibited co-immunoprecipitation. Heparitinase and pronase, but not chondroitinase ABC, degraded the CS receptors. CSFZ(Cys) (32 μg/ml) precipitated the purified proteoglycans as a high molecular weight smear (lane 1). Heparin (lane 2), but not chondroitin sulfate (lane 3) inhibited the precipitation. Pronase (lane 4) and heparitinase (lane 5), but not chondroitinase ABC (lane 6), degraded the high molecular weight band.

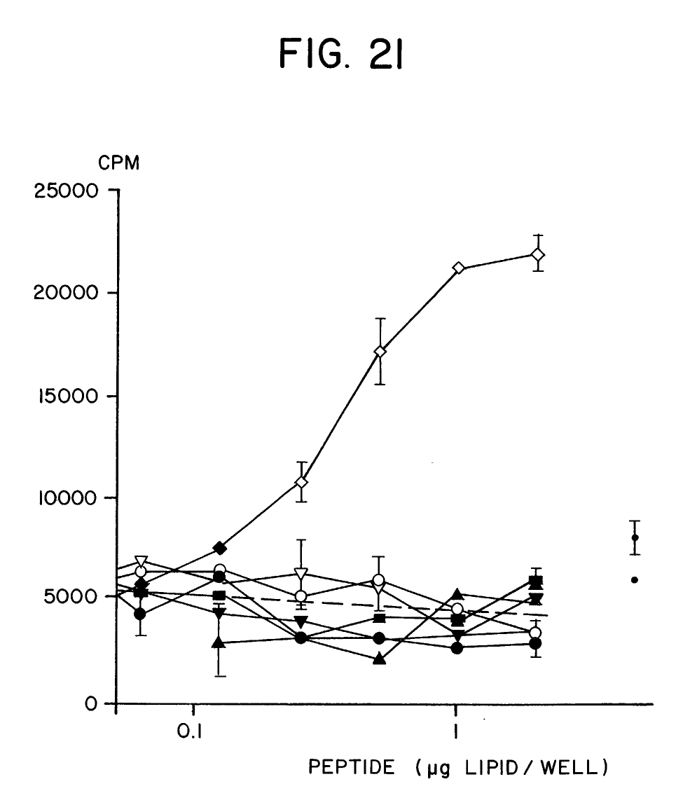

The peak labeled fractions from the anion exchange chromatography were pooled (22,500 cpm) and loaded onto a molecular sieve Superose 6 column in 50 mM Tris buffer pH 6.5 containing 7 M urea. The ³⁵SO₄ label eluted in a broad peak between 400 and 700 kDa as illustrated in FIG. 18. Thyroglobulin (669 kDa), apoferritin (443 kDa), β-amylase (200 kDa) and alcohol dehydrogenase (150 kDa) were used as molecular weight markers. Fractions (500 μl) were collected and counted. Fractions 13-22 (19,000 cpm, derived from 7×10⁷ HepG2 cells), and subjected to amino acid with hexosamine analysis. Results are shown in Table 4. On the basis of the amino acid analyses, the sample contained 2.8 μg of the proteoglycan.

By molecular sieving chromatography on a Superose 6 column, the purified HSPG eluted in a broad peak with an apparent molecular weight between 400 and 700 kDa as illustrated in FIG. 18. The results indicate the presence of large amounts of glucosamine, presumably originating from the heparan sulfate chains, and among the amino acids, a relatively high proportion of serine and glycine. See Table 4.

TABLE 4
Compositional Analysis of the Purified
Trypsin-released Hep52 Cell Proteoglycan

| Component | Molar % | Component | Molar % |
|---|---|---|---|
| Gal-NH₂ | 5.2 | Pro | 4.4 |
| Glc-NH₂ | 40.5 | Tyr | 0.7 |
| Asp or Asn | 2.3 | Val | 2.4 |
| Glu or Gln | 4.5 | Met | 1.2 |
| Ser | 8.7 | Cys | 0.0 |
| Gly | 12.8 | Ile | 1.9 |
| His | 0.3 | Leu | 3.4 |
| Arg | 0.9 | Phe | 1.1 |
| Thr | 3.4 | Lys | 1.6 |
| Ala | 4.8 | | |

Examples 13-20 indicate that the binding of CS constructs to tissue sections and to HepG2 cells is specifically inhibited by heparitinase treatment of the cells, and by the presence of heparin in the incubation medium. In hepatocyte extracts, the CS receptors are sulfated molecules of 400-700,000 Mr, which migrate as a smear on SDS polyacrylamide gels and are digested by heparitinase. The size of the individual GAG chains, and of the core protein, remain to be determined. After pronase digestion, the sulfated molecules are significantly reduced in size to about 200,000 Mr, perhaps representing remnants of the core protein linked to multiple GAG chains.

Biosynthetically labeled, sulfated molecules are rapidly removed from the surface membrane of HepG2 cells by low concentrations of trypsin, and more than 80% of the labeled released molecules are precipitated with the CS. The ³⁵SO₄-labeled molecules from the membrane of HepG2 cells were purified by two chromatographic steps. By molecular sieving chromatography in the presence of 7 M urea, their apparent molecular weight is between 400 and 700 kDa, and on a molar basis they contain 40% glucosamine, the amino sugar found in heparan sulfate GAG chains. The smaller amounts of galactosamine may be derived from O-linked oligosaccharides, or from chondroitin sulfate GAG chains, known to be present in syndecans. The amino acid analysis reflects only the composition of the trypsin-resistant portion of the core protein and reveals the presence of a high content of serine and glycine. GAGs are known to be assembled on serine residues of the core protein, frequently within the amino acid sequence acidic-X-Ser-Gly-acidic (Bourdon et al., P.N.A.S. USA, 84:3194-3198, 1987; Zimmerman and Ruoslahti, EMBO J. 8:2975-2981, 1989.

Heparitinase digestion or the presence of heparin in the incubation medium hinders CS binding to the receptor. The GAG chains are therefore involved in CS recognition, a conclusion further supported by the observation that the invasion of hepatocytes by sporozoites in vitro is inhibited by heparin, dextran sulfate and fucoidan. Nevertheless, a role for the core protein(s) in the interaction with the CS cannot be excluded.

HSPGs are ubiquitous constituents of mammalian cell surfaces, and they may behave as integral membrane proteins (Brandan and Hirschberg, J. Biol. Chem. 264:10520-10526, 1989; Kjellen et al., J. Biol. Chem. 255:10407-10413, 1980; Kjellen et al., P.N.A.S. USA, 78:5371-5375, 1981; Stow et al., J. Cell, Biol. 100:975-980, 1985), or peripheral membrane proteins which can be released from the cell surface by treatment with heparin or high ionic strength (Brandan and Hirschberg, J. Biol. Chem. 264:10520-10526, 1989; Kjellen et al., J. Biol. Chem. 255:10407-10413, 1980; Kjellen et al., P.N.A.S. USA 78:5371-5375, 1981; Oldberg et al., J. Cell. Biol. 100:975-980, 1979; Soroka and Farquhar, J. Cell, Biol. 113:1231-1241, 1991). Both types of HSPGs have been reported to be present on the hepatocyte surface exposed to the space of Disse. A third possible mode of membrane attachment is through a phosphatidylinositol (PI) anchor (Isihara et al., J. Biol. Chem. 262:4708-4716, 1987). However, this is unlikely to be the mode of attachment of the CS receptors, since in epithelial cells, PI-anchored molecules are sorted to the cell apical surface (Lisanti and Rodriguez-Boulan, TIBS, 15:113-118, 1990), while CS receptors are restricted to the basolateral cell membrane of hepatocytes. In fact, CS receptors are not cleaved from the membrane of HepG2 cells by PI-PLC, and are not eluted by treatment of the cells with heparin. As shown in FIG. 14, most sulfate-labeled CS receptors are released from subconfluent HepG2 cells by mild trypsinization. Therefore, the core protein of the HSPG is most likely attached to the membrane through a hydrophobic peptide anchor.

CS receptors share some properties with the syndecan family of HSPGs.

The following materials and methods were used in Examples 21-29 below.

Materials

Circumsporozoite proteins: The yeast-derived recombinant CS proteins Vivax-1 and -2, Falciparum-1, and -2 were obtained from the Chiron Corporation, Emeryville Calif.

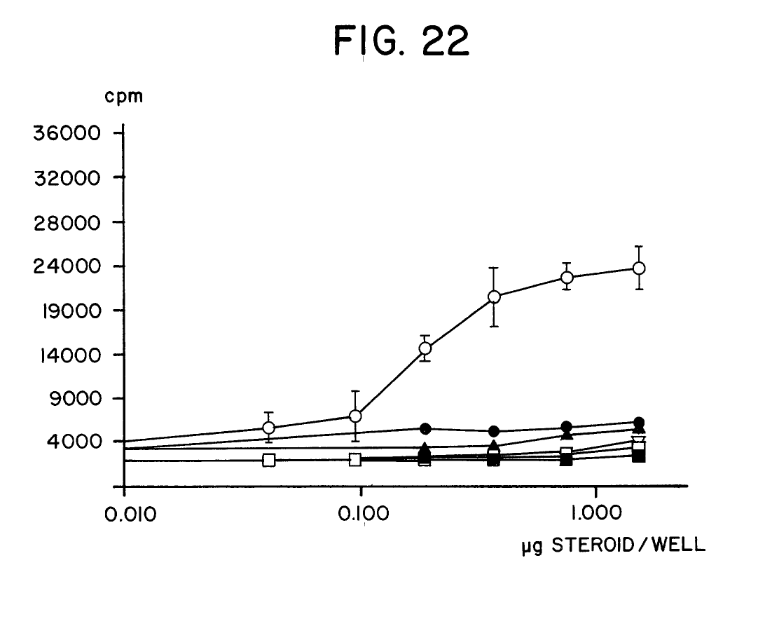

FIG. 19 illustrates the recombinant proteins Vivax-2, Falciparum-2, Vivax-1 and Falciparum-1 and their relation to the entire CS protein. The bottom half of the panel shows the sequence of Region II from P. vivax, P. falciparum and P. berghei. Amino acids shared by all three proteins are enclosed in boxes.

P. berghei sporozoites: Partially purified salivary gland P. berghei sporozoites were extracted for 10 minutes at room temperature with 0.5% NP-40, 150 mM NaCl, 25 mM Tris pH 8.8, and protease inhibitors: 1 mM PMSF, 0.25 μg/ml Pepstatin, 5 μg/ml Leupeptin, 5 μg/ml Aprotinin, 5 μg/ml Antipain (Boehringer Mannheim—Indianapolis, Ind.). After centrifugation, the supernatants were used in the solid phase binding assay described below. In some experiments, extraction was performed in the presence of 50 mM iodoacetamide (IA) to alkylate any free sulfhydryl groups in the CS protein.

Reduction and alkylation of CS proteins: Vivax-2 and P. berghei CS proteins were respectively reduced with 10 mM or 75 mM dithiothreitol (DTT) for 30 minutes at room temperature and alkylated with excess IA for 30 minutes at room temperature. The proteins were then passed through a Sephadex G-25 column to remove DTT and IA, and then assayed for binding to the various lipids and steroids.

Antibodies: The following monoclonal antibodies were used: 2F2, 2A10, and 3D11 (Yoshida et al. Science 207:71-73, 1980; Nardin, et al. J. Exp. Med. 156:20-30, 1982), which recognize the repeat-containing domains of P. vivax, P. falciparum, and P. berghei CS proteins respectively, and 2E6, which recognizes the liver stage of P. berghei. The rabbit antiserum to the peptide CSVTCGSGIRVRKRKGSNKKAEDL, reacting with thrombospondin, was used. The first six amino acids of the peptide consist of a motif shared by thrombospondin and Region II of the CS proteins.

Steroids, glycolipids and anionic polymers were obtained from Sigma (St. Louis, Mo.), except for 5-cholenic-3-sulfate.

Methods

Solid phase binding assays: Thirty μl of lipids or steroids dissolved in methanol (20-250 μg/ml) were dried into wells of microassay plates (Cat.# 3911—Falcon—Oxnard, Calif.). The plates were then blocked with 1% BSA/PBS pH 7.4 (BSA/PBS) for 30 minutes. Wells were incubated with recombinant protein (or with parasite extract) in PBS/BSA for 1 hour, washed 3 times with BSA/PBS, incubated for 1 hour with monoclonal antibodies 2F2 or 2A10 in BSA/PBS, washed 3 times, and then incubated with (10⁵ cpm/well) ¹²⁵I protein A (Amersham Corp., Arlington Hts., Ill.). In the case of the P. berghei CS protein, wells were incubated with ¹²⁵I labelled mAb 3D11. Wells were washed again and the bound radioactive protein was counted in an LKB gamma-counter (model #1260, Pharmacia, Inc., Gaithersburg, Md.). For inhibition assays, the recombinant proteins were mixed with dilutions of the various polymers in BSA/PBS, the mixture was loaded into the wells of the microtiter plates, and the binding assay performed as above. See also, Roberts, et al. J. Biol. Chem. 260:9405-9411, 1985.

Thin layer chromatography: The purity of the various lipids and steroids was analyzed by thin layer chromatography (plates 7011-04—J. T. Baker—Phillipsburg, N.J.) using the solvent system CHCl₃/MeOH/conc.NH₄OH, 80/20/0.4.

SDS-PAGE and Western blotting: SDS-PAGE was performed in 10% slab gels. For Western blotting, the gel contents were electrophoretically transferred to Immobilon (Millipore Corp. -Bedford, Mass. (Towbin, et al. P.N.A.S. USA 76:4350-4354, 1979. The membrane was then blocked for 1 hour with BSA/PBS, incubated for 1 hour with specific antibodies (mAbs or rabbit antisera), washed 3 times with PBS/0.5% Tween-20 and then incubated with goat anti-mouse or anti-rabbit Ig coupled to alkaline-phosphatase (Sigma) for 1 hour. The bound enzyme was developed with bromochlorophenol blue and nitrotetrazolium blue in 0.1 M Tris, 0.1 M NaCl, 5 mM MgCl₂.

Infection assay: Semiconfluent monolayers of HepG2 cells were grown in 10% FCS/MEM on 8 chamber slides (#4808 Lab-tek -Naperville, Ill.) for 24 hours prior to each experiment. Sporozoites were pre-incubated in media alone or with the various polymers for 15 minutes at 4° C. 5×10⁴ sporozoites, in a volume of 10 μl, were added per chamber. After 4 hours of incubation at 37° C., the medium containing the sporozoites was gently aspirated, and 0.5 ml MEM/10% FCS was added. Fresh medium was added at 18 hours and 28 hours, and then cells were fixed with methanol containing 0.3% H₂O₂ at 48 hours. After fixation, the wells were blocked with 10% FCS/PBS and were incubated with 10 μg/ml of mAb 2E6. Binding of the mAb was detected with a polyclonal goat anti-mouse Ig conjugated to horseradish peroxidase. The bound enzyme was revealed with 1 mg/ml 3,3′-diaminobenzidine in 50 mM Tris pH 7.6, 0.01% H₂O₂. The parasites in each well are counted under a 40× light microscope objective.

Immunofluorescence: P. berghei and P. falciparum sporozoites were dissected from mosquito salivary glands, fixed with 0.1% glutaraldehyde, and then dried into the wells of IFA slides. The sporozoites were incubated with polyclonal rabbit anti-CSVTCGSGIRVRKRKGSNKKAEDL sera (diluted 1:20 in PBS) for 1 hour at 37° C., washed with PBS, incubated with FITC-labeled goat anti-mouse in PBS for 1 hour at 37° C.

Circumsporozoite reaction: The P. berghei sporozoites were incubated for 30 minutes at room temperature with 5 μg/ml of mAb 3D11 in 10% FCS/PBS, in the presence or absence of 50 μg/ml dextran sulfate, and scored for the typical precipitate formation under the light microscope.

Iodination: The labelling of mAbs with ¹²⁵I was performed with Iodogen (Pierce, Rockford, Ill.) according to the manufacturer's instructions.

### EXAMPLE 21

Binding of CS Protein to Sulfatide and Cholesterol-3-Sulfate

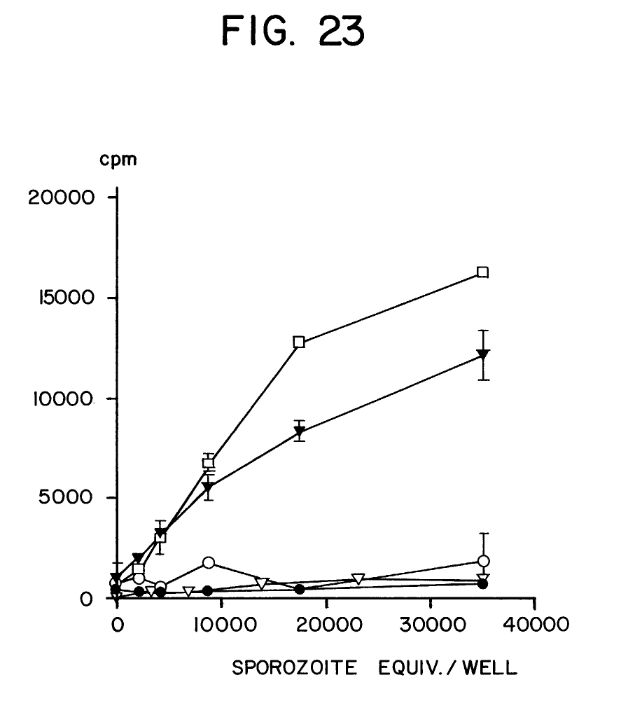

1.4 nmol of sulfatide (open squares), cholesterol-3-sulfate (closed triangles), galactocerebroside I (closed circles), galactocerebroside II (open circles) and cholesterol (open triangles) were evaporated into microtiter wells and then incubated with serial dilutions of Vivax-2. Binding was revealed with mAb 2F2 followed by iodinated protein A. Results are illustrated in FIG. 20. Vivax-2 bound to sulfatide and to cholesterol-3-sulfate, but not to the asialoganglioside or the galactocerebroside I and II.

### EXAMPLE 22

Binding of CS Proteins to Sulfatides But Not to Sulfatide Analogs

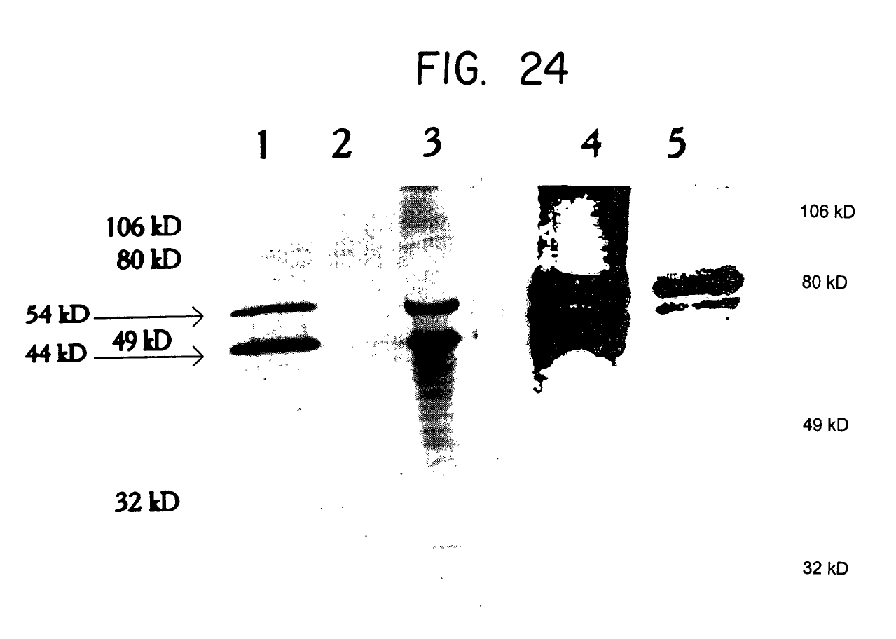

Serial dilutions of sulfatide (closed diamonds), trisialoganglioside (open circles), monosialoganglioside-GM1 (closed circles), monosialoganglioside-GM2 (open triangles), cholesterol (closed triangles-down), galactocerebroside I (open triangles-down), galactocerebroside II (closed triangles-up), disialoganglioside-GD1b (open squares), disialoganglioside-GD1a (closed squares) were evaporated onto microtiter wells and incubated with 0.15 μg/well of Falciparum-2. In all cases binding of the CS protein was revealed with mAb 2A10 followed by iodinated protein A. Results are illustrated in FIG. 21.

Falciparum-2, like Vivax-2, specifically bound sulfatide and not trisialoganglioside, monosialogangliosides-GM1 or GM2, galactocerebrosides type I or II, disialogangliosides-GD1a or GD1b, or cholesterol. Falciparum-1 (0.15 μg/ml) did not bind with sulfatide (small closed circles) and cholesterol-3-sulfate (small closed squares).

### EXAMPLE 23

Specificity of CS Binding to Cholesterol-3-Sulfate

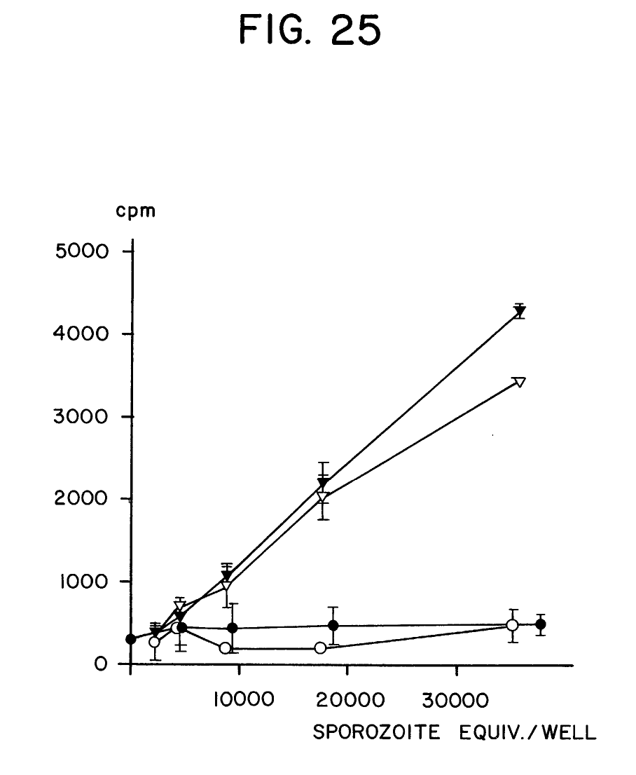

Serial dilutions of cholesterol-3-sulfate (open circles), cholesterol (closed circles), 5-cholenic acid-3β-ol (open triangles-up), 5-cholenic-3β-ol-sulfate (closed triangles-up), lithocholic acid (closed squares), lithocholic acid 3-sulfate (open squares), androsterone 3-sulfate (open triangles-down) and androsterone (closed triangles-down) were evaporated onto microtiter wells and incubated with 0.3 μg/well Vivax-2. The binding of the CS proteins was revealed with mAb 2F2. Results are illustrated in FIG. 22.

Vivax-2 bound to cholesterol-3-sulfate, and the binding increased proportionally to the amount of plastic-immobilized ligand. No binding was observed with the following analogues: cholesterol, 5-cholenic acid-3β-ol-sulfate, 5-cholenic acid-3β-ol, lithocholic acid-3-sulfate, lithocholic acid, androsterone sulfate (5-Androsten-3β-ol-17-one sulfate) and dehydroepiandrosterone (5-Androsten-3β-ol-17-one). To exclude the possibility that some negative results were due to the lack of binding of the compounds themselves to the plastic, the steroids were eluted with methanol from another set of identical wells, subjected to chromatography on TLC plates (CHCl₃/MeOH, 85/25), and revealed with phosphomolybdic acid. The various steroids were recovered from the wells at the expected concentrations.

### EXAMPLE 24

Binding of CS Protein to Sulfatide and Cholesterol-3-Sulfate

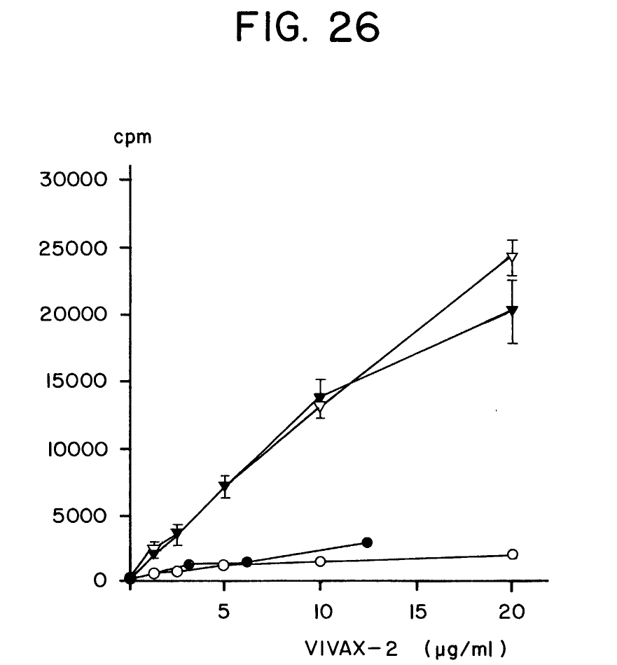

1.4 nmol of sulfatide (open squares), cholesterol-3-sulfate (closed triangles), galactocerebroside I (closed circles), galactocerebroside II (open circles) and cholesterol (open triangles) were evaporated into microtiter wells and then incubated with serial dilutions of P. berghei sporozoite extracts. Binding was revealed either with iodinated mAb 3D11 or with mAb 2F2 followed by iodinated protein A. Results are illustrated in FIG. 23. No binding to the non-sulfated analogues galactocerebrosides I and II, or to cholesterol was observed.

### EXAMPLE 25

Western Blot of CS Proteins to Sulfatides

In Western blots the P. berghei CS protein usually appears as 2 bands. The band of lower Mr is the membrane form, and the band of higher Mr may represent an intracellular precursor. Yoshida et al., J. Exo. Med. 154:1225-1236, 1981. NP-40 extracts of P. berghei sporozoites and Falciparum-2 were prepared in the presence of protease inhibitors and alkylating agents, and were incubated with sulfatide coated wells. The bound material was eluted with SDS sample-buffer and subjected to Western Blotting in the presence (lanes 1-3) and in the absence (lanes 4 and 5) of 2-ME. This material was also run on 10% SDS PAGE, transblotted, and revealed with mAbs 3D11 or 2A10, followed by goat anti-mouse linked to alkaline phosphatase.

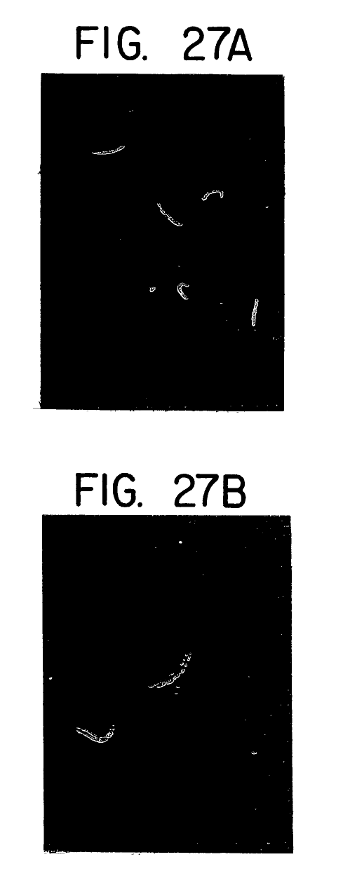

Western blots of total P. berghei extract (lane 3), P. berghei CS protein eluted from wells coated with sulfatide (lane 1) and disialoganglioside (lane 2) are illustrated in FIG. 24.

A similar experiment was performed with recombinant proteins, to exclude the possibility that aggregates, or a minor population of molecules, bound to the glycolipid. As also shown in FIG. 24, the Western blot patterns of the total preparation of Falciparum-2 (lane 4) and of the sulfatide-bound fraction (lane 5) are identical.

### EXAMPLE 26

Inhibition of CS Binding

Vivax-2 (0.12 μg/well) and P. berghei sporozoite extract (35,000 sporozoite equivalents/well) were mixed with inhibitor as illustrated in Table 5, and were incubated in wells coated with 1.4 nmol/well of sulfatide or cholesterol-sulfate. Binding was revealed with either mAb 2F2 or mAb 3D11, followed by iodinated protein A. Percent inhibition is based on the results of sextuplicate wells and calculated by comparison with wells in which no inhibitor was added.

TABLE 5
INHIBITION OF BINDING OF CS PROTEIN TO SULFATIDE
AND CHOLESTEROL-3-SULFATE

| | | | CONCEN- | | |
|---|---|---|---|---|---|
| | | | TRATION | | |
| BINDING | BINDING | | OF IN- | | |
| PROTEIN | SUBSTRATE | INHIBITOR | HIBITOR | | % INHIBITION |
| VIVAX-2 | SULFATIDE | DEXTRAN-SULFATE | 1 | μg/ml | 19 |
| | | | 10 | μg/ml | 70.2 |
| | | DEXTRAN | 1 | mg/ml | 0 |
| | | HEPARIN | 100 | μg/ml | 0 |
| | | HEPARAN-SULFATE | 1 | mg/ml | 0 |
| | | CHONDROITIN | 1 | mg/ml | 0 |
| | | SULFATE-A | | | |
| | | CHONDROITIN | 100 | μg/ml | 22.3 |
| | | SULFATE-B | 1 | mg/ml | 34.3 |
| | | CHONDROITIN | 1 | mg/ml | 0 |
| | | SULFATE-C | | | |
| | | KERATAN-SULFATE | 1 | mg/ml | 37 |
| | | HYALURONIC ACID | 1 | mg/ml | 0 |
| VIVAX-2 | CHOLESTEROL- | DEXTRAN-SULFATE | 1 | μg/ml | 12.2 |
| | SULFATE | | 10 | μg/ml | 21.1 |
| | | | 100 | μg/ml | 25.6 |
| | | | 1 | mg/ml | 33 |
| | | | 10 | mg/ml | 40.5 |
| | | DEXTRAN | 10 | mg/ml | 0 |
| | | HEPARIN | 1 | mg/ml | 0 |
| | | | 10 | mg/ml | 31.2 |
| | | HEPARAN-SULFATE | 10 | mg/ml | 0 |
| | | CHONDROITIN | 1 | mg/ml | 0 |
| | | SULFATE-A | 10 | mg/ml | 16 |
| | | CHONDROITIN | 100 | μg/ml | 0 |
| | | SULFATE-B | 1 | mg/ml | 27 |
| | | | 0 | mg/ml | 51 |
| | | CHONDROITIN | 10 | mg/ml | 0 |
| | | SULFATE-C | | | |
| | | KERATAN-SULFATE | 10 | mg/ml | 0 |
| | | HYALURONIC ACID | 1 | mg/ml | 0 |
| | | | 10 | mg/ml | 28 |
| P.berghei | SULFATIDE | DEXTRAN-SULFATE | 1 | μg/ml | 31.2 |
| CS protein | | | 10 | μg/ml | 63 |
| | | | 100 | μg/ml | 79 |

The binding of Vivax-2 and of the native P. berghei protein to sulfatide was inhibited by dextran-sulfate. Fifty percent inhibition of binding was achieved at dextran-sulfate concentrations between 5 and 10 μg/ml. The inhibition was specific, and not simply a consequence of electrostatic interactions. The charged and sulfated compounds chondroitin-sulfate A, chondroitin-sulfate C, dermatan sulfate, heparan-sulfate, keratan-sulfate and hyaluronic acid had no effect at concentrations of 1 mg/ml, while chondroitin-sulfate B was only slightly inhibitory at 1 mg/ml.

In contrast to the above findings, the binding of Vivax-2 to cholesterol-3-sulfate was poorly inhibited by dextran-sulfate. Although a clear dose-dependence was observed, less than 50% inhibition was achieved at 10 mg/ml of this compound. Other compounds, however, were even less inhibitory, indicating specificity of the interactions of the CS protein receptors and the CS protein or ligands.

### EXAMPLE 27

Binding of CS Protein to Sulfatides and Cholesterol-3-Sulfate Is Dependent on the Integrity of a Disulfide Bond in Region II

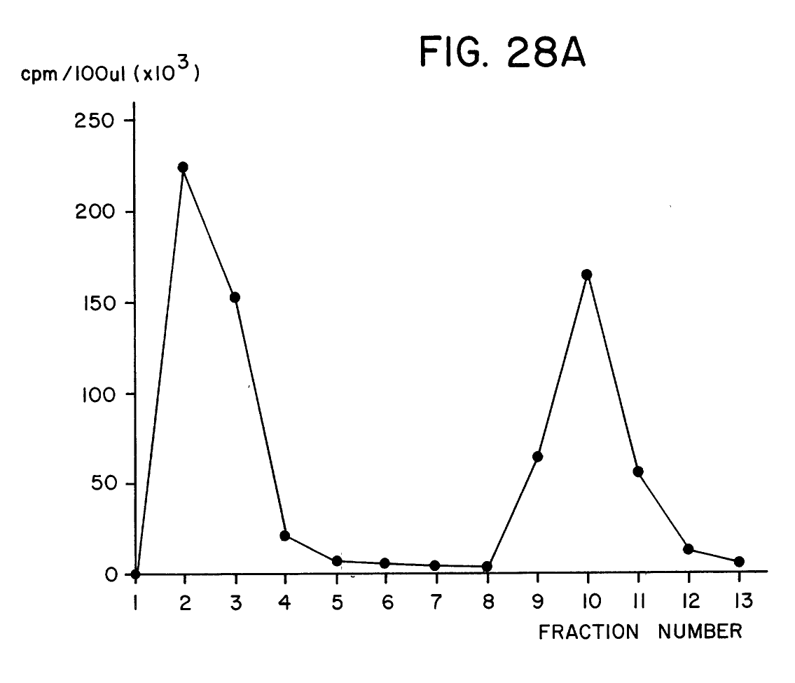

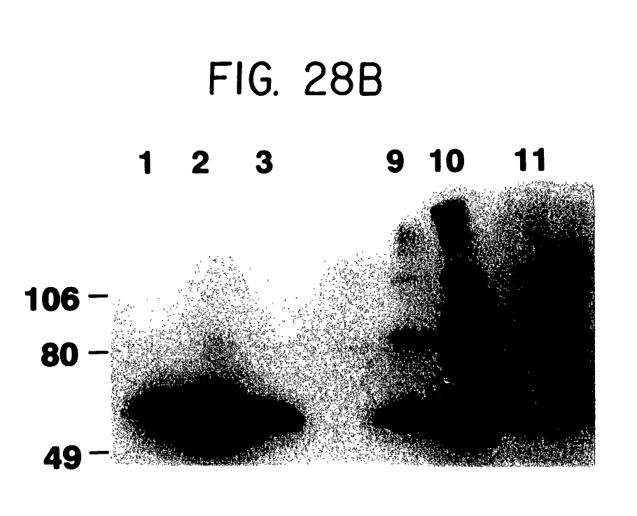

P. berghei sporozoite extract and Vivax-2 were reduced and alkylated with dithiothreitol and iodoacetamide (circles), alkylated with iodoacetamide alone (open triangles), or not treated (closed triangles) and then incubated in wells coated with either cholesterol-sulfate (closed circles) or sulfatides (open circles, open and closed triangles). Binding was revealed either with iodinated mAb 3D11 or with mAb 2F2 followed by iodinated protein A. The results illustrated in FIGS. 25 and 26 demonstrate that reduction and alkylation abolished recognition of both sulfatide and cholesterol-3-sulfate, but alkylation alone did not.

### EXAMPLE 28

Region II+ of the CS Protein Is Exposed on the Surface of Sporozoites

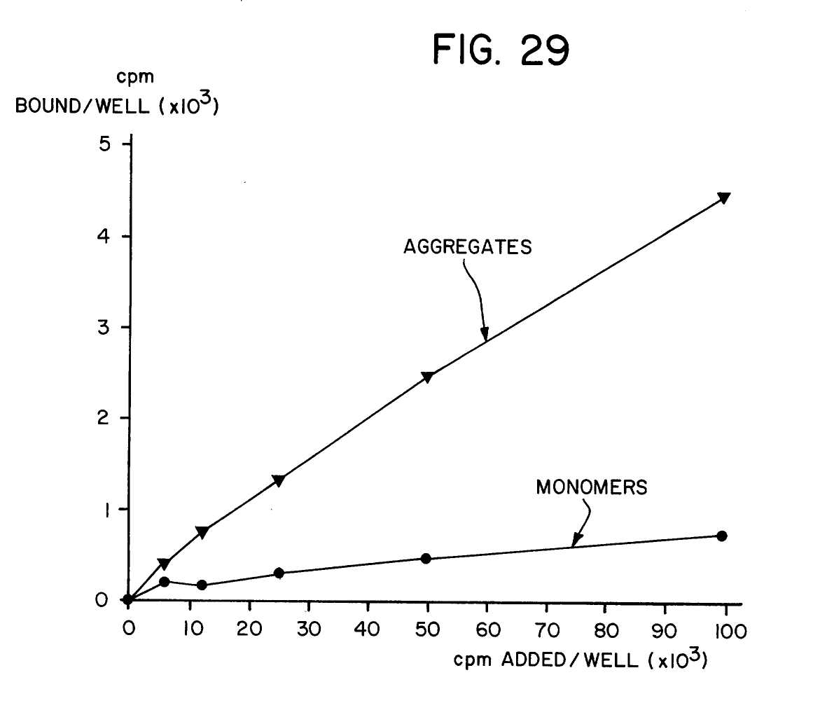

Glutaraldehyde-fixed P. falciparum (FIG. 27A) and P. berghei (FIG. 27B) sporozoites were incubated with anti-CSVTCGSGIRVRKRKGSNKKAEDL peptide antisera followed by goat anti-rabbit-Ig FITC-labeled antibody, and observed with a fluorescence microscope. This antisera reacted with glutaraldehyde-fixed P. berghei and P.falciparum sporozoites as well as with live P. berghei sporozoites.

### EXAMPLE 29

Dextran Sulfate Inhibition of Sporozoite Invasion

HepG2 cells were plated at a density of 0.5×10⁶, infected with P. berghei sporozoites (50,000/well) in the presence of dextran, dextran-sulfate (molecular weight 5,000), chondroitin-sulfate A, heparan-sulfate or media alone and then incubated for 48 hours. The cells were fixed and stained with mAb 2E6, followed by goat-anti-mouse linked to horseradish peroxidase. Results are illustrated in Table 6. Numbers represent schizonts counted/20 fields under 40× magnification in a light microscope.

TABLE 2
EFFECT OF DEXTRAN-SULFATE ON SPOROZOITE INVASION

| | | CONCENTRATION | PARASITE |
|---|---|---|---|
| RUN# | INHIBITOR | (μg/ml) | #** |
| 1 | MEDIUM | — | 186, 231 |
| | DEXTRAN-SULFATE | 50 | 37, 91 |
| | DEXTRAN | 50 | 169, 203 |
| | CHONDROITIN- | 50 | 214, 220 |
| | SULFATE A | | |
| 2 | MEDIUM | — | 269, 196 |
| | DEXTRAN-SULFATE | 50 | 24, 78 |
| | | 5 | 90, 187 |
| | DEXTRAN | 50 | 323, 216 |
| | | 5 | 251, 235 |
| | CHONDROITIN- | 50 | 266, 197 |
| | SULFATE A | 5 | 250 |
| 3 | MEDIUM | — | 311, 316 |
| | DEXTRAN-SULFATE | 50 | 81, 85 |
| | | 5 | 226, 275 |
| | HEPARAN-SULFATE | 50 | 255, 254 |
| | | | 265, 237 |
| 4 | MEDIUM | — | 218, 191 |
| | DEXTRAN-SULFATE | 50 | 0, 0 |
| | | 5 | 51, 105 |
| | DEXTRAN | 50 | 251, 223 |
| 5 | MEDIUM | — | 237, 217 |
| | DEXTRAN-SULFATE | 50 | 0, 0 |
| | DEXTRAN | 50 | 218, 234 |
| 6* | MEDIUM | — | 144, 138 |
| | DEXTRAN-SULFATE | 50 | 84, 120 |
| | DEXTRAN | 50 | 158, 110 |
| 7* | MEDIA | — | 210, 211 |
| | DEXTRAN-SULFATE | 50 | 216, 200 |
| | DEXTRAN | 50 | 233, 206 |

*In these experiments dextran-sulfate and dextran were added to the culture four hours after the addition of the parasites, after completion of invasion.
**Number of exoerythrocytic liver stages in 20 microscope fields (40X). Each experiment was performed in duplicate.

As illustrated in Table 6, dextran-sulfate (mol wt 5,000) inhibited invasion in a dose-dependent fashion, while at similar doses dextran, chondroitin-sulfate A and heparan-sulfate had no effect. If the dextran-sulfate was added to the medium after 4 hours of contact between the sporozoites and the HepG2 cells (i.e., after invasion had occurred), the development of the liver stages was not affected.

To discern possible toxic effects of dextran sulfate on the sporozoites, the CSP reaction was performed on the treated parasites. This reaction is only observed in infective sporozoites, and can be used as a crude measurement of viability. Sporozoites which had been incubated for 1 hour at 37° C. in 100 FCS/PBS containing 50 μg/ml of dextran sulfate, developed CSP reactions in the same proportion and with similar intensity as sporozoites incubated in medium alone.

Several proteins share one or more copies of the motif Cys-Ser-Val-Thr-Cys-Gly-X-X-X-X-X-Arg-X-Arg (Roberts, et al. J. Biol. Chem. 260:9405-9411, 1985; Holt et al., J. Biol. Chem. 264:12138-12140, 1988; Robson et al. Nature 335:79-82, 1988; Goundis et al Nature 335:82-85, 1988; Clarke, et al. Mol. Biochem. Parasitol. 41:269-280, 1990. These include proteins with seemingly unrelated functions, such as the adhesive molecules thrombospondin and von Willebrand factor; the complement protein properdin; PyPSSP2, another sporozoite protein; terminal complement components F-spondin and UNC-5; the leech anticoagulant antistasin, the coat proteins of sporozoites (the CS protein) and of Herpes simplex I virus; and two non-characterized proteins, TRAP from the blood stage of malaria parasites and EtHL6 from Eimeria. Several of these proteins bind specifically to sulfatide, suggesting that the common motif recognizes sulfated glycoconjugates.

Recombinant proteins Vivax-2 and Falciparum-2 representing large segments of the P. vivax and P. falciparum CS molecules, as well as the native P. berghei CS protein, bind not only to sulfatide but also to cholesterol-3-sulfate. These interactions are ligand-specific since the proteins do not bind to structurally related gangliosides, neutral lipids or to other negatively charged (sulfated or non-sulfated) steroids.

It is also believed that the recognition of sulfatide and cholesterol-sulfate involves the shared amino acid motif, because only the recombinant proteins Vivax-2 and Falciparum-2, but not Vivax-1 or Falciparum-1, bind to sulfatide and cholesterol sulfate. The binding and non-binding recombinant proteins differ only at their C-terminal end: Vivax-2 and Falciparum-2 have an additional 25 amino acids, 14 of which (Region II) contain the motif shared with the other sulfatide-binding proteins.

Examples 21-29 indicate that the motif itself binds both to sulfatide and to cholesterol-3-sulfate. As shown in Table 5, dextran sulfate inhibited sulfatide binding to CS at much lower concentrations than those required to inhibit cholesterol-3-sulfate binding, perhaps reflecting the lower avidity of the latter compound for CS.

While not intending to be bound by any theory, applicants believe that recognition of cholesterol-3-sulfate by CS, but not by other sulfatide-binding proteins may be explained by the cluster of basic residues at the C-terminal end of the motif interacting with SO4⁻. This may require the correct spatial orientation of the ligand, provided by its interaction with the other amino acids in the motif. Perhaps only the side chains of the CS-specific amino acids in the motif can correctly position cholesterol-3-sulfate in the binding site.

The inhibitory effect of dextran-sulfate is observed only if dextran-sulfate is present during incubation of sporozoites with the target cells, but not if it is added to the incubation medium after invasion, suggesting that the polymer acts in the initial steps of recognition of the target cells. Although a non-specific toxic effect of dextran sulfate on sporozoites can not be excluded, the polysaccharide did not affect the CSP which is only observable with variable parameters.

A variety of hematopoietic cell lines bind to microtiter plates coated with Vivax-2 but not to Vivax-1, and Region II (and in particular the VTCG sequence) appears to be involved in this interaction. All these observations strongly suggest that the receptor of CS (and of the motif) is a sulfated molecule.

It has also been observed that the staining pattern of in vivo bound CS27IVC was identical to that in frozen liver sections incubated in vitro with CS27IVC. In vivo bound CS27IVC closely followed the contours of the liver sinusoids, suggesting accumulation in the Disse space. However, there appears to be more selectivity in vivo than in vitro. Centrilobular veins appear negative in vivo labelling but positive in in vitro labelling. While not intending to be bound by any theory, these observations could be explained by the absence of fenestrae in the centrilobular vein epithelium. Therefore, the underlying hepatocytes are not accessible to CS27IVC in circulation in the sinusoids.

Furthermore, the biological properties of glycosaminoglycan chains in different organs may depend upon both their structural features and their availability to the relevant ligands.

Cleavage products of the heparan sulfate proteoglycans are typically prepared by trypsin or protease cleavage or degradation. These cleavage products are not the same as the entire proteoglycan but retain the ability to bind the ligands or mimetics described above.

Liver Cell Targeting

Minutes after mosquitoes inject malaria sporozoites into mammalian hosts the parasites enter hepatocytes, strongly suggesting that target cell recognition by the parasite is receptor-mediated. GAG chains of HSPG from the basolateral side of hepatocytes have binding affinity for the Region II+ of CS protein, and synthetic peptides representing Region II+ inhibit the invasion of HepG2 cells by certain sporozoites. The remarkable target cell specificity of malaria sporozoites suggests new approaches for the development of inhibitors to prevent malaria infection and for the delivery of substances to hepatocytes.

The following materials and methods were used in Examples 30-32.

Material

Recombinant Proteins: CS27IVC and Falc-1 are described above.

Antibodies: The monoclonal antibody mAb 2A10 (Nardin et al., J. Exp. Med. 156:20, 1982) is directed against an epitope contained in the (NNP), repeat domain of the P. falciparum CS.

Mice: Balb/C males, from Taconic Farms, weighing between 15 and 20 grams were used.

Methods

Fluorescein Isothiocyanate (FITC) Labeling of mAb 2A10: The antibody labeling was performed as in Harlow et al., Antibodies. A Laboratory Manual, Cold Spring Harbor, Cold Spring Harbor Laboratories (1988).

⁵¹Chromium labeling of red blood cells (RBC): Mouse blood (200 82 l) was collected from the retroorbital sinus, and washed with phosphate buffered saline (PBS), pH 7.4 containing 1% BSA (BSA/PBS). The RBC pellet was incubated with 10 μCi of Na⁵¹CrO₄ for 30 minutes, and then washed with BSA/PBS.

¹²⁵-Iodine labelling: The recombinant proteins were labeled using iodogen to a specific activity of −2.5×10⁵ cpm/μg. To reduce the amount of oxidative damage to the proteins, 1 mCi of 125I in 10 μl of 0.1M Na phosphate buffer was oxidized in a glass tube pre-coated with iodogen for 5 minutes. Ten μl were then transferred to another tube, and incubated for 5 minutes on ice with 10 μl (10 μg) of CS27IVC of Falc-1. The free iodide was removed by filtration in Sephadex G-25 (Isolab Inc., Akron, Ohio), and dialysis against 50 mM Tris, 75 mM NaCl, pH 7.4 (buffer A).

Isolation of CS27IVC Multimers: Radiolabeled CS27IVC was applied to a 1 ml Heparin-Sepharose column (Sigma Chemical C., St. Louis, Mo.) which had been preequilibrated with 50 mM Tris, 75 mM NaCl, 1 BSA, 0.05% Tween-20 (Biorad, Hercules, Calif.) pH 7.4 (buffer B), and washed with 5 column volumes of the same buffer. The bound material was eluted with 1 ml 50 mM Tris, 1.5 M NaCl, pH 7.4, and dialyzed against buffer A.

Binding of CS to HepG2 Cells: 10⁵ HepG2 cells (ATCC number HB8065, Rockville, Md.) were deposited in 96 microtiter wells of Removawell plates (Dynatech Laboratories, Chantilly, Va.) and grown overnight in minimum essential medium with 10% fetal calf serum (Gibo, Grand Island, N.Y.), 1 mM L-glutamine (Gibco), 3 mg/ml glucose (Sigma), 1× nonessential amino acids (Gibo), 50 μg/ml penicillin, and 100 μg/ml streptomycin (Gibco). The cells were fixed with 4% paraformaldehyde, washed three times with TBS (25 mM Tris-Cl, pH 7.4, 138 mM NaCl), and stored at 4° C. in buffer B. The wells were incubated with serial dilutions of the iodinated proteins for 1 hour, washed three times with buffer B, and counted in an LKB gamma counter. Cerami et al., Cell 70:1021 (1992)

Clearance Studies: Mice were anesthetized with ether, and injected with 10⁵ cpm of ¹²⁵I-labeled CS27IVC or Falc-1 via the periorbital sinus. At two, five and fifteen minutes after injection, the mice were exsanguinated, the organs removed, rinsed in TBS, blotted dry on filter paper, and counted for radioactivity. To estimate the amounts of blood contaminating the various organs, we repeated the same procedure in mice injected with 100 μl (1.5×10⁵ cpm) of ⁵¹Cr-labeled RBC. The mean volumes of blood in the liver, spleen and kidney of the exsanguinated animals were 75, 24, and 35 μl respectively. In all other organs, the amount of blood was negligible, i.e., less than 15 μl. The percent of injected dose of CS which was retained in the various organs was calculated as described as follows:

$$\% retained cpm = \frac{corrected cpm per organ}{total injected dose} \times 100$$

after subtracting the CS counts from the contaminating blood. The total blood volume was calculated as in Little et al. Biology of Laboratory Mouse, New York: Dover Publications 1956.

In other experiments, light and electron microscopy were used to localize CS in the various organs. The mice were injected i.v. with 100 μg of unlabeled CS27IVC or Falc-1 via the retroorbital plexus. The mice were exsanguinated five minutes after injection, the organs removed, rinsed in TBS, blotted dry, and then snap frozen in liquid nitrogen for light microscopy. For electron microscopy, the organs were cut into small pieces and fixed in a mixture of 0.1% glutaraldehyde and 4% paraformaldehyde.

Light Microscopy: The frozen tissue was embedded in Tissue Tek O.C.T. (Miles Inc., Naperville, Ill.) and cut into 5 μm sections. Sections were dried for 30 minutes, fixed for 10 minutes in 4% paraformaldehyde, and either used immediately or stored at 4° C. in PBS containing 1% BSA, 0.5% Tween-20. After blocking with the same buffer, the sections were incubated with mAb 2A10 conjugated to FITC, and examined in a fluorescence microscope.

Electron Microscopy: The fixed liver specimens were dehydrated in ethanol and embedded in Lowicryl K4M at −20° C. (Frevert et al., Infect. and Immun. 80:2349 (1992)). After UV polymerization, sections were cut with a RMC MT-7 ultramicrotome and stained by sequential incubation with 15 μg/ml mAb 2A10 and protein A gold 10 nm (PAG10) (1:30, Amersham, Arlington, Ill.). Cerami et al., Cell 70:1021 (1992). Control sections were strained only with the gold conjugate. Photographs were taken with a Zeiss EM 910 electron microscope.

### EXAMPLE 30

Isolation of CS27IVC Multimers by Affinity Chromatography on Heparin-Sepharose

CS multimers bind to the GAG chains of HSPG and were isolated by affinity chromatography on heparin-sepharose.

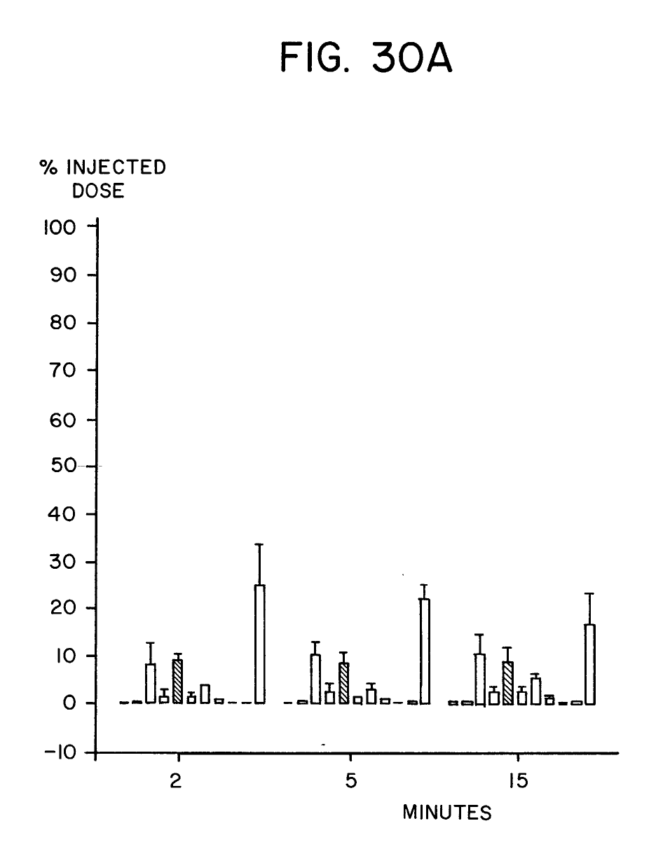

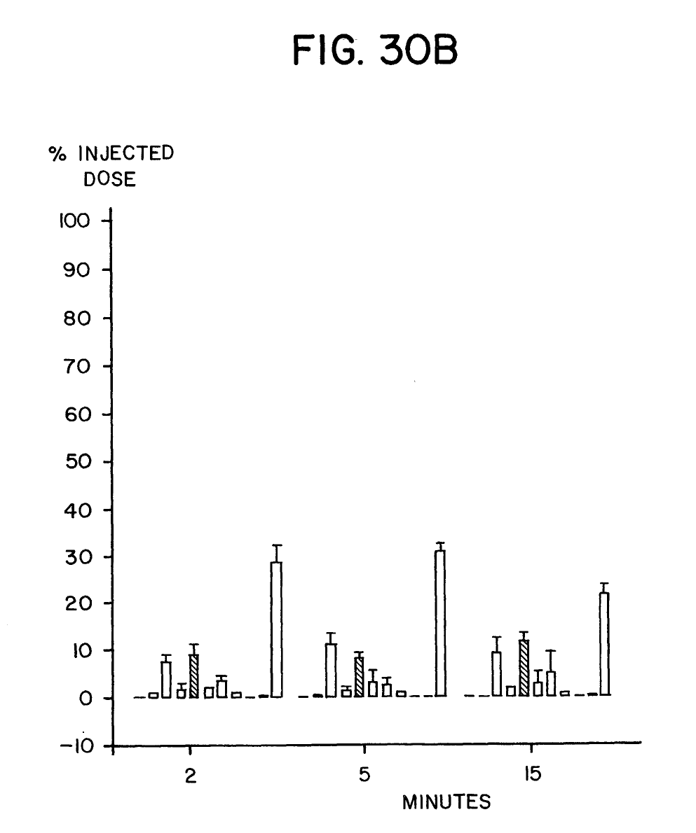

¹²⁵I-labeled CS27IVC was applied to a heparin-sepharose column pre-equilibrated in buffer A. The column was washed with 5 ml of the same buffer, and the bound CS27IVC was eluted with 50 mM Tris, 1.5 M NaCl, pH 7.4. The ordinates represent the cpm in 10 μl of each fraction (FIG. 28A). Ten μl of selected fractions were run on 10% SDS-PAGE under non-reducing conditions, the gel was dried and subjected to radioautography (FIG. 28B).

As shown in FIG. 28A, about 30% of the radiolabeled CS27IVC bound to the heparan-sepharose column, and the bound molecules were eluted with a buffer containing a high salt concentration. SDS-PAGE analysis under non-reducing conditions showed that the break-through peak contained only CS monomers, while the heparin-binding material contained various CS multimers or aggregates as well as monomers (FIG. 28B). Under reducing conditions, a single band corresponding to a CS monomer was detected in proteins under both peaks, indicating that the CS multimers consisted of mixed aggregates of disulfide linked dimers, trimers, etc. and non-covalently bound monomeric CS.

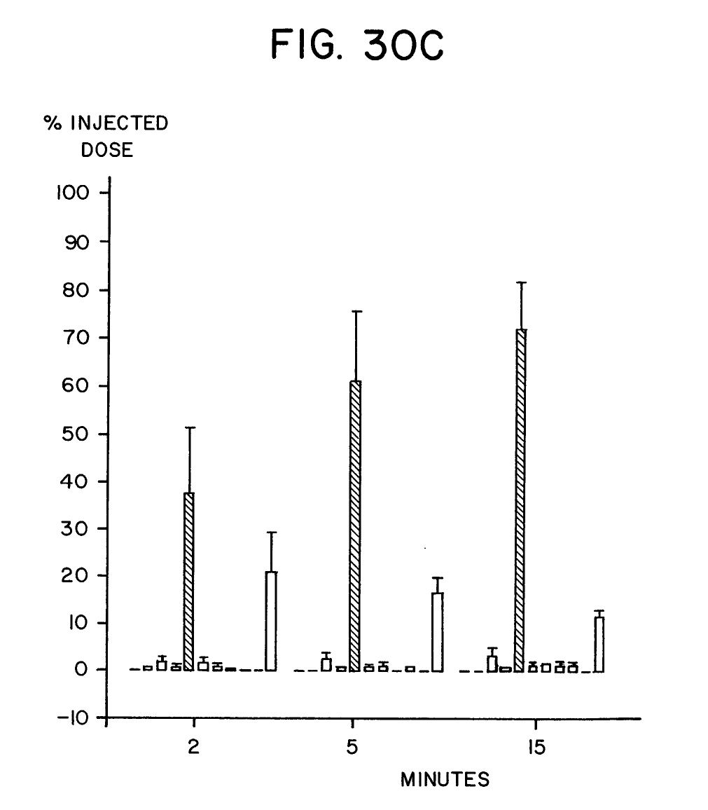

Fractions 2-4 (monomers), and 9-11 (aggregates) were pooled and dialyzed against 50 mM Tris, 75 mM NaCl. Serial dilutions ere then incubated with fixed HepG2 cells for 30 minutes at room temperature. The wells were washed, and the remaining counts measured in a gamma counter (FIG. 29).

FIG. 29 shows that the CS27IVC fractions obtained by filtration in sizing columns (Cerami et al., Cell 70:1021 (1992)), and by affinity chromatography on heparin-sepharose have similar properties, i.e., only the CS multimers or aggregates bind to HepG2 cells.

### EXAMPLE 31

Clearance of CS27IVC and Falc-1

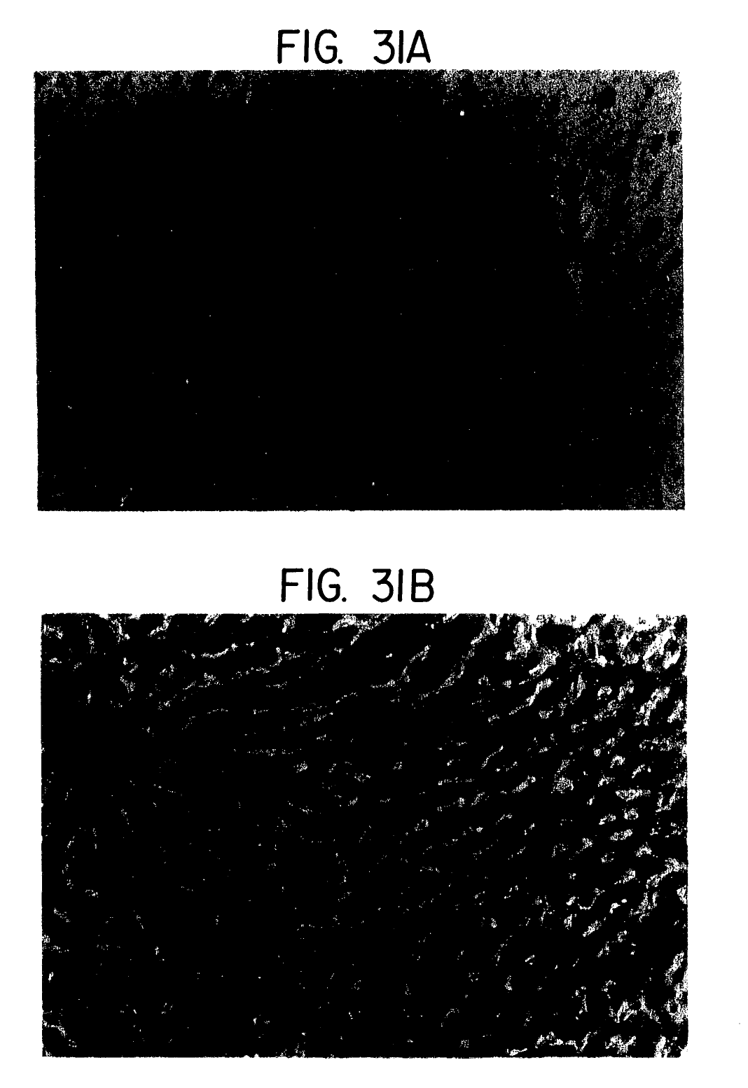

Radiolabeled Falc-1 (FIG. 30B), and the aggregated (FIG. 30C) and monomeric (FIG. 30A) fractions of radiolabeled CS27IVC proteins were injected intravenously into the retroorbital sinuses of mice. The animals were sacrificed 2, 5 and 15 minutes later, and the amounts of CS in the blood and in various organs were measured. Additionally, the radioactivity associated with the various organs was calculated. As shown in FIGS. 30A-C (From left to right, the bars represent cpm associated with: bladder, heart, two kidneys, large intestines, liver, two lungs, small intestine, stomach, spleen, thyroid. The last bar represents cpm in total blood volume. Each bar represents mean volumes±S.D. of cpm in organs form three mice.), there were differences in the pattern of clearance of multimers of CS27IVC, as compared to that of Falc-1, or of the monomers of CS27IVC. Only about 40% of the injected CS monomers, or of Falc-1 counts were recovered in the blood and in various organs between 2 and 15 minutes after injection, and 4% or less were associated with the liver at any time (FIGS. 30A and 30B). In sharp contrast, 80% or more of the injected multimer CS counts were recovered, and most were in the liver, i.e., 45% at 2 minutes, 55% at 5 minutes, and 70% at 15 minutes. There was practically no accumulation of CS multimers in the bladder, heart, kidney, large intestine, liver, lung, small intestine, stomach, spleen or thyroid (FIG. 30C).

### EXAMPLE 32

The CS27IVC Aggregates Accumulate on the Microvilli of Hepatocytes

Mice were injected with 100 μg CS27IVC or Falc-1, and 5 minutes later they were killed. The livers and kidneys were removed, washed in TBS, blotted dry and snap frozen in liquid nitrogen. Frozen tissues were stained with mAb 2A10 conjugated to FITC.

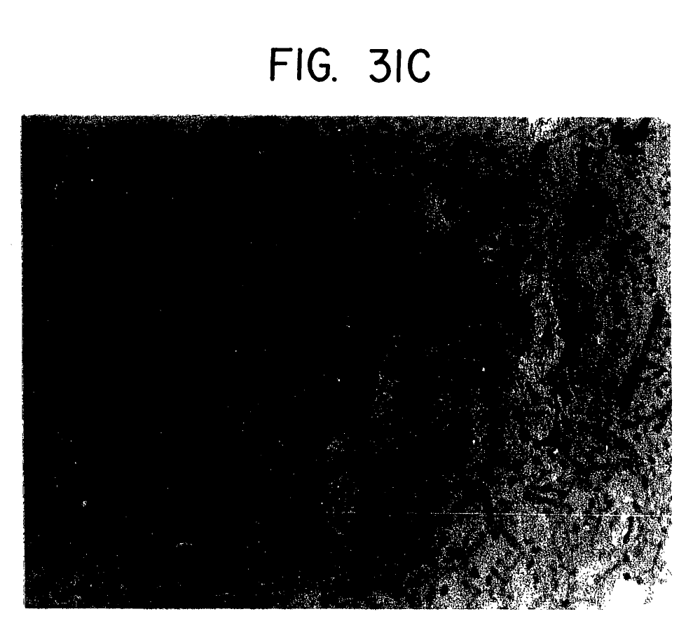

No staining with FITC-labeled mAb 2A10 was seen in the livers (FIG. 31A) or kidneys of mice injected with Falc-1. In animals injected with CS27IVC, the recombinant was readily detected in the liver (FIG. 31B), but not in the kidney (FIG. 31C) or other organs. In the liver, the bound CS closely followed the contours of the sinusoids, suggesting accumulation in the Disse space. The staining pattern was identical to that previously observed in frozen sections incubated in vitro with the same recombinant CS. Cerami et al., Cell 70:1021 (1992).

Mouse liver was fixed 15 minutes after intravenous injection of 100 μg CS27IVC. The tissue was embedded in Lowicryl K4M, and sections were stained with mAb 2A10 and protein A-coated gold particles PAG10.

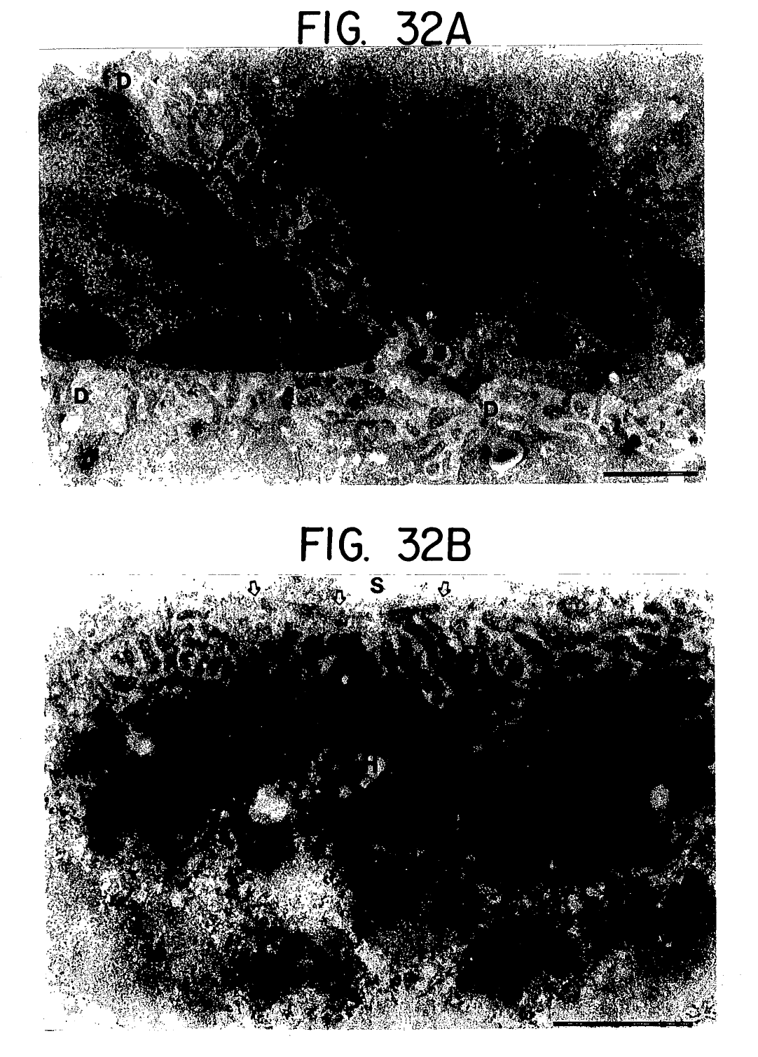

By electron microscopy, the gold label was detected in the space of Disse (D) of the liver (FIG. 32A). There was no label on a Kupffer cell (K) in the sinusoidal lumen (S). Single or doublets of gold particles were found in close association with the microvilli of hepatocytes (H) (FIG. 32B). No gold was found on the endothelial cell surface facing the sinusoidal lumen (S) (arrows) (M=mitochondrium; Bars−1 μm) or Kupffer cell surfaces.

Accordingly, cleavage products of the heparan sulfate proteoglycan of an hepatocyte or a mimetic thereof can be utilized to target or deliver a substance to an hepatocyte as described above.

What is claimed is:

1. A peptide inhibitor for the binding of a circumsporozoite polypeptide to receptors of hepatocytes from malaria-susceptible mammals, said inhibitor having an amino acid sequence selected from the group consisting of:
(i) Region II+ of a circumsporozoite protein, said Region II+ containing the subsequence CSVTCG;
(ii) fragments of said Region II+ containing at least a portion of the adhesion ligand for said receptors, said portion comprising at least one cysteine of said Region II+;
(iii) peptide constructs comprising (a) (i) or (ii) and (b) at least one other fragment of the amino acid sequence of said circumsporozoite protein, said constructs having no substantial ability to elicit the formation of antibodies recognizing the immunodominant epitope of said circumsporozoite protein.

2. A peptide inhibitor for the binding of a circumsporozoite polypeptide to basolateral plasma membrane of hepatocytes from malaria-susceptible mammals, said inhibitor having an amino acid sequence selected from the group consisting of:
(i) Region II+ of a circumsporozoite protein, said Region II+ containing the subsequence CSVTCG;
(ii) fragments of said Region II+ containing at least a portion of the adhesion ligand for said receptors, said portion comprising at least one cysteine of said Region II;
(iii) peptide constructs comprising (a) (i) or (ii) and (b) at least one other fragment of the amino acid sequence of said circumsporozoite protein, said constructs having no substantial ability to elicit the formation of antibodies recognizing the immunodominant epitope of said circumsporozoite protein.

3. A method of inhibiting the binding of a circumsporozoite polypeptide to hepatocytes susceptible to sporozoite invasion, said method comprising:
supplying to the environment of said hepatocytes, the peptide inhibitor of claim 1 in an amount effective to inhibit said binding, no later than exposure of said hepatocytes to said circumsporozoite protein.

4. The method of claim 3, wherein said circumsporozoite polypeptide is native circumsporozoite protein present on the surface of viable, infectious malarial sporozoites.

5. The method of claim 3, wherein said circumsporozoite polypeptide is selected from the group consisting of native circumsporozoite protein, recombinant circumsporozoite protein, and recombinant fragments of circumsporozoite protein each comprising Region II+.

6. The method of claim 3, wherein said hepatocytes are human hepatocytes.

7. A peptide consisting essentially of Region II+ of the circumsporozoite protein.

8. The peptide of claim 7, said Region II+ being selected from the group consisting of
E W S P C S V T C G N G I Q V R I K P G S A;
E W T P C S V T C G V G V - - R V R;
E W S P C S V T C G S G I R A R R K;
E W S P C S V T C G K G V R M R R K;
E W S P C S V T C G K G V R M R R K;
E W S P C S V T C G S G I R A R R K;
E W S Q C N V T C G S G I R V R K R;
E W S Q C S V T C G S G V R V R K R; and
E W S E C S T T C D E G R K I R R R;

9. The peptide of claim 8, said peptide being P C S V T C G N G I Q V R I K P G S A.

10. The peptide of claim 7, said peptide being P C S V T C G N G I Q V R I K.

11. The peptide of claim 8, said peptide being P C S V T C G V G V R V R.

12. An inhibitor for the binding of a circumsporozoite polypeptide to a receptor of an hepatocyte from a malaria susceptible mammal, said inhibitor comprising a mimetic of the inhibitor of claim 1.

13. An inhibitor for the binding of a circumsporozoite polypeptide to a receptor of an hepatocyte from a malaria susceptible mammal, said inhibitor comprising a mimetic of the inhibitor of claim 2.

14. An inhibitor for the binding of a circumsporozoite polypeptide to a receptor of an hepatocyte from a malaria susceptible mammal, said inhibitor comprising a mimetic of the peptide of claim 7.

15. A method of inhibiting the binding of a circumsporozoite polypeptide to hepatocytes susceptible to sporozoite invasion, said method comprising:
supplying to the environment of said hepatocytes, the mimetic of claim 12 in an amount effective to inhibit said binding, no later than exposure of said hepatocytes to said circumsporozoite protein.

16. The method of claim 15, wherein said circumsporozoite polypeptide is native circumsporozoite protein present on the surface of viable, infectious malarial sporozoites.

17. The method of claim 15, wherein said circumsporozoite polypeptide is selected from the group consisting of native circumsporozoite protein, recombinant circumsporozoite protein, and recombinant fragments of circumsporozoite protein each comprising Region II+.

18. The method of claim 22, wherein said hepatocytes are human hepatocytes.

19. A method of delivering a substance to a hepatocyte in a mammal, said method comprising:
combining said substance with an inhibitor as defined in claim 1 to yield an inhibitor/substance complex; and
administering said complex to said mammal.

20. The method of claim 19, wherein said substance comprises DNA.

21. The method of claim 19, wherein said substance comprises a pharmaceutically active compound.

22. A method of delivering a substance to a hepatocyte in a mammal, said method comprising:
combining said substance with an inhibitor as defined in claim 2 to yield an inhibitor/substance complex; and
administering said complex to said mammal.

23. The method of claim 22, wherein said substance comprises DNA.

24. The method of claim 22, wherein said substance comprises a pharmaceutically active compound.

25. A method of delivering a substance to a hepatocyte in a mammal, said method comprising:
combining said substance with a peptide as defined in claim 7 to yield an inhibitor/substance complex;
administering said complex to said mammal.

26. The method of claim 25, wherein said substance comprises DNA.

27. The method of claim 25, wherein said substance comprises a pharmaceutically active compound.

28. A method of delivering a substance to a hepatocyte in a mammal, said method comprising:
combining said substance with a mimetic as defined in claim 12 to yield an mimetic/substance complex; and
administering said complex to said mammal.

29. The method of claim 28, wherein said substance comprises DNA.

30. The method of claim 28, wherein said substance comprises a pharmaceutically active compound.

31. An inhibitor for the binding of circumsporozoite polypeptide or a polypeptide as defined in claim 1 to a receptor of an hepatocyte from a malaria susceptible mammal, said inhibitor comprising a cleavage product of a heparan sulfate proteoglycan from the surface of said hepatocyte.

32. A method of inhibiting the binding of a circumsporozoite polypeptide to hepatocytes susceptible to sporozoite invasion, said method comprising:
supplying to the environment of said hepatocytes, the cleavage product of claim 31 in an amount effective to inhibit said binding, no later than exposure of said hepatocytes to said circumsporozoite protein.

33. The method of claim 32, wherein said circumsporozoite polypeptide is native circumsporozoite protein present on the surface of viable, infectious malarial sporozoites.

34. The method of claim 32 wherein said circumsporozoite polypeptide is selected from the group consisting of native circumsporozoite protein, recombinant circumsporozoite protein, and recombinant fragments of circumsporozoite protein each comprising Region II+.

35. The method of claim 32, wherein said hepatocytes are human hepatocytes.

36. An inhibitor for the binding of a circumsporozoite polypeptide or a polypeptide as defined in claim 1 to a receptor of an hepatocyte from a malaria susceptible mammal, said inhibitor comprising a mimetic of a cleavage product of a heparan sulfate proteoglycan from the surface of said hepatocyte.

37. A method of inhibiting the binding of a circumsporozoite polypeptide to hepatocytes susceptible to sporozoite invasion, said method comprising:
supplying to the environment of said hepatocytes, the peptide inhibitor of claim 36 in an amount effective to inhibit said binding, no later than exposure of said hepatocytes to said circumsporozoite protein.

38. The method of claim 37, wherein said circumsporozoite polypeptide is native circumsporozoite protein present on the surface of viable, infectious malarial sporozoites.

39. The method of claim 37, wherein said circumsporozoite polypeptide is selected from the group consisting of native circumsporozoite protein, recombinant circumsporozoite protein, and recombinant fragments of circumsporozoite protein each comprising Region II+.

40. The method of claim 37, wherein said hepatocytes are human hepatocytes.

41. A method of delivering a substance to a hepatocyte in a mammal, said method comprising:
combining said substance with an inhibitor as defined in claim 31 to yield an inhibitor/substance complex; and
administering said complex to said mammal.

42. The method of claim 41, wherein said substance comprises DNA.

43. The method of claim 41, wherein said therapeutic agent comprises a pharmaceutically active agent.

44. A method of delivering a substance to a hepatocyte in a mammal, said method comprising:
combining said substance with an inhibitor as defined in claim 36 to yield an inhibitor/substance complex; and
administering said complex to said mammal.

45. The method of claim 44, wherein said substance comprises DNA.

46. The method of claim 44, wherein said therapeutic agent comprises a pharmaceutically active agent.
